(19) United States
(12) Patent Application Publication
(10) Pub. No.: US 2004/0202994 A1
(43) Pub. Date: Oct. 14, 2004

(54) Apparatus and method for on-chip concentration using a microfluidic device with an integrated ultrafiltration membrane structure

(75) Inventor: Aaron T. Timperman, Morgantown, WV (US)

Correspondence Address: PALMER & DODGE, LLP, PAULA CAMPBELL EVANS, 111 HUNTINGTON AVENUE, BOSTON, MA, 02199, US

(73) Assignee: West Virginia University Research Corporation

(21) Appl. No.: 10/784,393

(22) Filed: Feb. 23, 2004

Related U.S. Application Data
(60) Provisional application No. 60/449,282, filed on Feb. 21, 2003.

(51) Int. Cl. C12Q 1/00; C12M 1/34

(52) U.S. Cl. 435/4; 435/288.5; 435/288.7; 435/297.2

(57) ABSTRACT

The present invention provides a microfluidic device capable of reacting an enzyme or other agent with a substantially purified polypeptide. In one embodiment of the present invention, the microfluic device comprises a plurality of reaction channels wherein the substantially purified polypeptide is delivered to a reaction channel. Once confined within the reaction channel, the substantially purified polypeptide engages the enzyme or agent and produces a reaction product. In a preferred embodiment of the present invention, the reaction product is concentrated at a charged membrane prior to being removed from the microfluidic device. Additionally, the present invention provides a method of concentrating a positive analyte at a positively charged membrane and a negative analyte at a negatively charged membrane. Once concentrated, the analytes may be removed from the microfluidic device.

### RELATED APPLICATIONS

This application claims the benefit of U.S. Provisional Application No. 60/449,282, filed on Feb. 21, 2003. The entire teachings of the above application(s) are incorporated herein by reference.

### FIELD OF THE INVENTION

The present invention provides a microfluidic system and microfluidic devices for proteome analysis and methods for making and using the same. More specifically, the present invention provides an apparatus and method of concentrating a sample with the aid of an ultrafiltration membrane on a microfluidic device prior to subsequent analysis.

### BACKGROUND OF THE INVENTION

The goal of proteomics is to identify and quantitate all of the proteins expressed in a cell as a means of addressing the complexity of biological systems. Current methods for proteome analysis generally are based on the use of two-dimensional electrophoresis (“2DE”) to identify cellular proteins. Protein patterns on 2DE gels are analyzed using image analysis techniques to generate proteome maps. Proteome maps of normal cells and diseased cells are compared to detect proteins that are up- or down-regulated during physiological responses to disease. These proteins are excised for identification and characterization, using such methods as mass fingerprinting and mass spectrometry.

However, using current 2DE methods, only the most abundant proteins can be identified. Thus, most of the proteins identified by 2DE methods represent structural proteins or housekeeping proteins. These problems have limited the use of proteomics for the identification of cancer markers because the lower abundance proteins that produce aberrant cell signals cannot be qualified, making it difficult to elucidate mechanisms that cause disease states and identify suitable cancer-specific markers.

The lack of sensitivity of current 2DE-based technology is caused primarily by a lack of separating or resolving power because high abundance proteins mask the identification of low abundance proteins. Loading more protein on the gels does not improve the situation because the Gaussian tails of the high abundance spots contaminate the low abundance proteins. The use of zoom gels (2D gels that focus on a narrow pH range) allows for minimal gains but is considered too cumbersome to be of any practical utility. Selective enrichment methods also can be used but generally at the expense of obtaining a comprehensive view of cellular protein expression. The sensitivity of detection on 2DE gels also is problematic, because the amount of protein required for identification by mass spectrometry (“MS”) is near the detection limits of the most sensitive methods for visualization of the protein spots on the 2DE gels. Further, the polyacrylamide matrix typically used in 2DE gives rise to a significant amount of background in the extracted sample mixture making subsequent analysis by MS difficult. Additionally, during peptide extraction following typical in-gel digestion procedures, the sample is exposed to many surfaces and losses can be substantial, particularly for low abundance proteins (Timperman, 2000, Anal. Chem. 72: 4115-4121a).

Multi-dimensional column separations offer many advantages over 2DE, including a higher separating power and reduced sample contamination and loss. A typical large format 2DE gel is capable of achieving a peak capacity of about 2,000 while 2D column separations can achieve peak capacities of over 20,000 for protein separations. Additionally, the stationary phases of these columns are very stable and non-reactive compared to polyacrylamide gels, leading to reduced sample contamination and loss. Many different types of separation techniques have been coupled to 2D column separations including size exclusion, reversed phase chromatography, cation-exchange chromatography, and capillary electrophoresis. Further increases in peak capacity have been achieved using three-dimensional columns.

Microfluidic devices are finding many applications for DNA analysis, but there has been little development of these devices for protein analysis. The microfluidic device revolution was begun by Harrison, 1992, Analytical Chemistry 64: 1926-1932, who demonstrated valveless electrophoretic separation and fluid manipulation on such devices. Much recent work has focused on the basics of sample injection, on-device column fabrication and interfacing with mass spectrometry.

Miniaturized analysis systems based on microfluidic platforms have experienced explosive growth in research in the past decade. This rapid growth is due to the great potential that these systems offer in fields, such as analytical chemistry, clinical analysis, high-throughput screening, genomics, and proteomics. Some of the characteristics of miniaturized analysis systems that make them promising include: the ability to handle nL-pL volumes without dilution, low reagent consumption, and extremely fast separations. Flow can be controlled in complex microfluidic networks without the need for valves through the application of voltages and the use of electrokinetic and electroosmotic transport.

However, detection often remains a challenge for microfluidic systems as their inherently small dimensions limit the number of molecules available for detection or down-stream processing. A related separation technique, capillary electrophoresis, uses the same voltage driven mass transport as many microfluidic devices, and with similar dimensions, the detection challenges for CE are identical to those for microfluidic systems. To address detection problems in both microfluidic systems and capillary electrophoresis, much research has focused on on-chip and on-column sample concentration. Most concentration methods that have been developed for microfluidic devices are based on sample focusing, solid phase extraction, or field gradient methods.

Sample focusing and field amplification techniques require the use of a discontinuous buffer system and manipulate differences in the local electric field to focus ions at the interface between the two solutions. Field amplified sample stacking was first reported by Mikkers et al., and Chien, Burgi et al. who used this technique for capillary electrophoresis and furthered its abilities. Integration of field amplification stacking into a microfluidic device has been achieved and concentration factors up to 100-fold have been achieved.

Sample concentration has also been achieved in microfluidic devices and CE using solid-phase extraction. Solid phase extraction pre-concentration devices have been made with membranes, porous polymer monoliths, and beads. Solid phase extraction has been shown to be an efficient means of concentration, but recovery is dependent on the properties of the analytes and it requires buffer or solvent exchange.

Several field gradient and flow balancing methods have been reported as well. In these methods bulk flow of solution is counterbalanced by an external field gradient that opposes the bulk flow. These methods can produce impressive concentration factors, but need to be adjusted so that the forces balance each other. Additionally they require the use of hydrodynamic flow on the chip which can complicate flow control in channel networks. Both electric field gradient and temperature gradient methods have been used for analyte concentration. The temperature gradient method reported by Ross and Locascio has produced the highest reported concentration factors, up to 10,000-fold, for a broad range of individually concentrated analytes.

Isoelectric focusing is frequently used for the separation and concentration of proteins in larger gels, capillary columns, and microfluidic devices. This technique focuses protein along a pH gradient in the presence of an external electric field. Proteins are focused where the local pH is equal to their pI, and unfortunately proteins are less soluble at this pH. An interesting form of isoelectric focusing has been performed in tapered capillaries and microfluidic devices which uses a temperature gradient to produce a pH gradient. Thus, the pH gradient is formed in situ without the use of ampholytes or gels.

Membranes and other materials have been employed for size based analyte concentration. Dialysis membranes and gels have been used in CE. Another method for CE utilized acid etching of a section of the glass capillary that would allow current flow but was not permeable to large molecules. In this method, electroosmotic flow was still present and could remove the molecules being concentrated from the focusing section of the capillary. Ramsey et al. incorporated a porous frit that was polymerized between the bottom and top cover plate of a microfluidic device. Previously, this frit material was reported to produce irregular pores with diameters on the order of 300-500 nm. Concentration factors of 100-fold were observed for large DNA molecules, and small amounts of DNA became imbedded in the frit. A similar approach for polymerization of ultrafiltration membranes with smaller pores and a narrow pore size distribution in microfluidic devices is not straightforward.

Despite these advances, there is a need in the art to extend the application of concentrating small molecules in an electrically driven microfluidic system using ultrafiltration membranes of nanometer diameters.

In addition, there is a need in the art to incorporate a concentration system in a microfluidic chip that is efficient, flexible, and can be easily multiplexed. Also, there is a need in the art for a passive concentration device which does not require tuning or force balancing and can concentrate all anionic or cationic species in front of the membrane.

### SUMMARY OF THE INVENTION

The present invention provides a system and method for rapidly analyzing large numbers of compounds or complex mixtures of compounds, particularly low abundance cellular proteins involved in cell signaling pathways. The system may also be used to analyze analyte mixtures other than peptides including, but not limited to, organics in dissolved organic matter sample from natural waters and organic matter from coal. The system comprises a number of modular components which can be used in an integrated fashion, or separately, or, in conjunction with other systems.

The present invention provides a microfluidic device capable of reacting an enzyme or other agent with a substantially purified polypeptide. In one embodiment of the present invention, the microfluidic device comprises a plurality of reaction channels wherein the substantially purified polypepetide is delivered to a reaction channel. Once confined within the reaction channel, the substantially purified polypeptide engages the enzyme or agent and produces a reaction product. In a preferred embodiment of the present invention, the reaction product is concentrated at a charged membrane.

In a preferred embodiment of the present invention, the reaction channel comprises a first side channel and a second side channel. Additionally, the first side channel comprises a positively charged membrane and the second side channel comprises a negatively charged membrane. In a preferred embodiment, a positively charged analyte of the reaction product is concentrated at the positively charged membrane; additionally, a negatively charged analyte of the reaction product is concentrated at the negatively charged membrane. Following the reaction and concentration of the respective analytes, the concentrated analytes are removed form the microfluidic device.

In a preferred embodiment of the present invention, the system is driven by electroosmotic flow. In a preferred embodiment, a negative electrode is positioned adjacent to the positively charged membrane and a positive electrode is positioned adjacent to the negatively charged membrane. In a preferred embodiment, a charge trapping mechanism is developed at each membrane allowing analytes to be concentrated at a membrane wherein the diameter of the analyte is smaller than the diameter of the pore of the membrane. Such a result cannot be achieved with a hydrodynamic flow system. In one embodiment, a nanocapillary array (or a nanochannel array) is utilized to concentrate the respective analytes.

In one embodiment of the present invention, the above-described microfluidic device is integrated into an integrated proteomic analysis system. In another embodiment, various pieces of the integrated proteomic analysis system are repeated or omitted (i.e., integrating a first microfluidic device and a second microfluidic device or omitting a downstream separation module).

The present invention also provides a microfluidic device wherein a membrane is positioned between a first cover channel slide and a second cover channel side. Such a configuration provides a vertical channel in which to input a substantially purified polypeptide. In a preferred embodiment, a reaction product is concentrated before being removed from the microfluidic device.

The present invention provides a method of concentrating analytes on a microfluidic device. In a preferred embodiment, a substantially purified polypeptide is delivered to a microfluidic device. The substantially purified polypeptide is then delivered to a reaction channel wherein a positive analyte and a negative analyte are produced. In a preferred embodiment, the positive analyte is concentrated in front of a positively charged membrane and the negative analyte is concentrated in front of the negatively charged analyte. Once concentrated, the concentrated positive analyte and the concentrated negative analyte are removed from the microfluidic device.

Additionally, the present invention provides a method of analyzing a substantially purified polypeptide. In a preferred embodiment of the present invention, a substantially purified polypeptide is delivered to a microfluidic device. A first portion of the substantially purified polypeptide is delivered to a reaction channel. A second portion of the substantially purified polypeptide (unreacted) is delivered directly to a peptide analysis module for immediate testing. Once the first portion has undergone a reaction in the reaction channel, a reaction product is produced and delivered to the peptide analysis module for testing. A comparison between the test result generated from the unreacted portion two with a test result from the reacted portion one may produce valuable information regarding various properties of the substantially purified polypeptide.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1A shows an integrated proteome analysis system according to one aspect of the invention. FIG. 1B shows a cross-sectional view through an on-line digestion microfluidic device according to one aspect of the invention. FIG. 1C shows a cross-sectional view through an on-line digestion microfluidic device according to one aspect of the invention wherein one reaction channel does not comprise an agent.

FIG. 2A shows an etched microfluidic device according to another aspect of the invention comprising a plurality of reservoir wells. FIG. 2B shows an example of a microfluidic device of the present invention wherein reaction channels are in a parallel configuration. FIG. 2C chows an example of a microfluidic device of the present invention wherein the plurality of reaction channels are in a perpendicular configuration.

FIG. 3 shows a system for optimizing sample transport in a microfluidic device according to one aspect of the invention.

FIG. 4 is a schematic showing the connection between an interfacing microfluidic device and electrospray capillary according to one aspect of the invention.

FIG. 5 shows a cross-sectional view through an on-line digestion microfluidic device of the present invention wherein the microfluidic device engages a first electrode at a first end of a reaction channel and a second electrode at a second end of the reaction channel.

FIGS. 6A, 6B, 6C and 6D show an embodiment of the present invention in which a negatively charged protein enters the microfluidic system wherein the microfluidic system comprises side channels and further comprises a main channel with high electroosmotic flow.

FIGS. 7A, 7B, 7C, and 7D show an embodiment of the present invention in which a positively charged protein enters the microfluidic system wherein the microfluidic system comprises side channels and further comprises a main channel with high electroosmotic flow.

FIGS. 8A, 8B, 8C, and 8D shows an embodiment of the present invention wherein a main channel has been coated to create low electroosmotic flow and the anionic and cationic analytes are separated before entering the main channel.

FIG. 9 shows a microfluidic device of the present invention.

FIGS. 10A and 10B shows a fluid path of a microfluidic device of the present invention. FIGS. 10C, 10D, and 10D show methods to construct the embodiment of the present invention illustrated in FIGS. 10A and 10B.

FIG. 11 shows the concentration of 0.1 mM fluoroscein in front of a 10 nm pore ultrafiltration membrane of the present invention.

FIG. 12 shows an intensity profile of the fluorescence intensity for the images shown in FIG. 11.

FIG. 13 shows the concentration of FITC labeled peptides having different electrophoretic mobilities of the present invention.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides a system and method for rapidly analyzing large numbers of compounds or complex mixtures of compounds, particularly low abundance cellular proteins involved in cell signaling pathways. The system may also be used to analyze analyte mixtures other than peptides including, but not limited to, organics in dissolved organic matter sample from natural waters and organic matter from coal. The system comprises a number of modular components which can be used in an integrated fashion, or separately, or, in conjunction with other systems.

In one aspect of the present invention, a membrane was integrated into a microfluidic device for the purpose of concentrating analytes. In a preferred embodiment of the present invention, a nanocapillary array (or nanochannel array) is integrated into a microfluidic device for the purpose of concentrating analytes. Through the application of an electric field across the channel, charged analytes were concentrated in front of the membrane, and a concentrated analyte band was ejected from the channel by reversing the polarity of the electric field. In one aspect of the present invention, concentration factors up to 300-fold was measured. In one aspect of the present invention, a plurality of analytes can be concentrated in front of the same membrane without adjustments, provided that they are all anionic or cationic. In one aspect of the present invention, in the presence of an electric field a charge trapping effect was observed; small molecules can be concentrated in front of membranes with pore sizes which are orders of magnitude above the molecular weight cut-offs for hydrodynamically driven systems. Additionally, the concentrator places minimal requirements on the buffer system and is easily multiplexed.

For electrically driven concentration, analyte retention in front of the membrane appears to occur primarily by a charge trapping mechanism. In the presence of such a charge trapping mechanism, relatively large pores can be used to concentrate the small molecules, making the system more robust. The only known limitation to the buffer system is that the conductivity must be low enough to prevent current breakdown, and there is no need for multiple buffers or solvents which most concentrating for microfluidics and capillary electrophoresis require. Not only can this device be used for analyte concentration, but it can be used as a concentrating micro-reactor as many species can be co-localized in front of the membrane. With this simple and robust design, concentration factors of 300-fold have been achieved.

The invention further provides a method for proteome analysis comprising a system described above or one or more modules of the system. In a first step, in a preferred embodiment, a sample comprising a plurality of cellular polypeptides is contacted with the upstream separation module and polypeptides within the sample are separated to obtain a plurality of substantially purified polypeptides. A selected substantially purified polypeptide (e.g., a sample band) is delivered to a microfluidic module comprising the protease immobilized therein, and the polypeptide is exposed to the protease for a period of time and under conditions sufficient to substantially digest the polypeptide, thereby producing digestion products or peptides. In a preferred embodiment of the present invention, a positively charged reaction product (positive analyte) is concentrated at a positively charged membrane and a negatively charged reaction product (negative analyte) is concentrated at a negatively charged analyte. Once concentrated, the digestion products are transported to a downstream separation module where they are separated, and the substantially separated digestion products are delivered to the interfacing microfluidic module which transports the substantially separated digestion products to the peptide analysis module. The amino acid sequences of the digestion products are determined and assembled to generate the sequence of the polypeptide. Prior to delivery to the peptide analysis module, the interfacing module can perform one or more additional steps of separating, concentrating, and or focussing.

The steps of separating, producing digestion products, and analyzing digestion products to determine protein sequence, can be performed in parallel and/or iteratively for substantially all of the polypeptides of a sample to obtain a proteome map of a cell from which the polypeptides were obtained. Proteome maps from multiple different cells can be compared to identify differentially expressed polypeptides in these cells. In a particularly preferred embodiment, polypeptides which are differentially expressed in abnormally proliferating cells, such as cancer cells, are identified. Still more preferably, the polypeptides are cell signaling polypeptides. Molecular probes which specifically recognize differentially expressed polypeptides or nucleic acids encoding these polypeptides can be arrayed on a substrate to provide reagents to assay for the presence or absence of these polypeptides and/or nucleic acids in a sample.

Definitions

The following terms and definitions are used herein:

As used herein, a “substantially purified polypeptide” refers to a polypeptide sample which comprises polypeptides of substantially the same molecular mass (e.g., greater than about 90%, preferably greater than about 95%, greater than about 98%, and up to about 100% of the polypeptides in the sample are of substantially the same molecular mass). Substantially purified polypeptides do not necessarily comprise identical polypeptide sequences.

As used herein, “substantially the same molecular mass” refers to polypeptides which have a less than a 10 kdalton difference in molecular mass, preferably, less than a 5 kdalton difference in molecular mass, and most preferably, less than a 1 kd difference in molecular mass.

As used herein, a “biological fluid” includes blood, plasma, serum, sputum, urine, cerebrospinal fluid, lavages, and leukapheresis samples.

As defined herein, a “configuration of parallel channels” is one which provides a common voltage output at an intersection point between the channels. However, the geometric arrangement of the channels is not necessarily parallel. However, they should be configured as a set of parallel resistors in a circuit having a common input channel and a common output channel.

As used herein, a channel which has a geometric configuration which is “substantially parallel” to another is a channel which is at a less than 5 degree angle with respect to the longitudinal axis of the other channel. A channel which is “substantially perpendicular” another is a channel which is at a 90° angle with respect to the longitudinal axis of another channel, +/−5°.

As used herein, “a system processor” refers to a device comprising a memory, a central processing unit capable of running multiple programs simultaneously, and preferably, a network connection terminal capable of sending and receiving electrical signals from at least one non-system device to the terminal. The system processor is in communication with one or more system components (e.g., modules, detectors, computer workstations and the like) which in turn may have their own processors or microprocessors. These latter types of processors/microprocessors generally comprise memory and stored programs which are dedicated to a particular function (e.g., detection of fluorescent signals in the case of a detector processor, or obtaining ionization spectra in the case of a peptide analysis module processor, or controlling voltage and current settings of selected channels on a device in the case of a power supply connected to one or more devices) and are generally not directly connectable to the network. In contrast, the system processor integrates the function of processors/microprocessors associated with various system components to perform proteome analysis as described further below.

As used herein, “pathway molecules” or “pathway biomolecules” are molecules involved in the same pathway and whose accumulation and/or activity and/or form (i.e., referred to collectively as the “expression” of a molecule) is dependent on other pathway molecules, or whose accumulation and/or activity and/or form affects the accumulation and/or activity or form of other pathway target molecules. For example, a “GPCR pathway molecule” is a molecule whose expression is affected by the interaction of a GPCR and its cognate ligand (a ligand which specifically binds to a GPCR and which triggers a signaling response, such as a rise in intracellular calcium). Thus, a GPCR itself is a GPCR pathway molecule, as is its ligand, as is intracellular calcium.

As used herein a “peptide” refers to a biomolecule comprising fewer than 20 consecutive amino acids.

As used herein, a “polypeptide” refers to a biomolecule which comprises more than 20 consecutive amino acids. The term “polypeptide” is meant to encompass proteins, but also encompasses fragments of proteins, or cleaved forms of proteins, or partially digested proteins which are greater than 20 consecutive amino acids.

Integrated Proteomic Analysis System

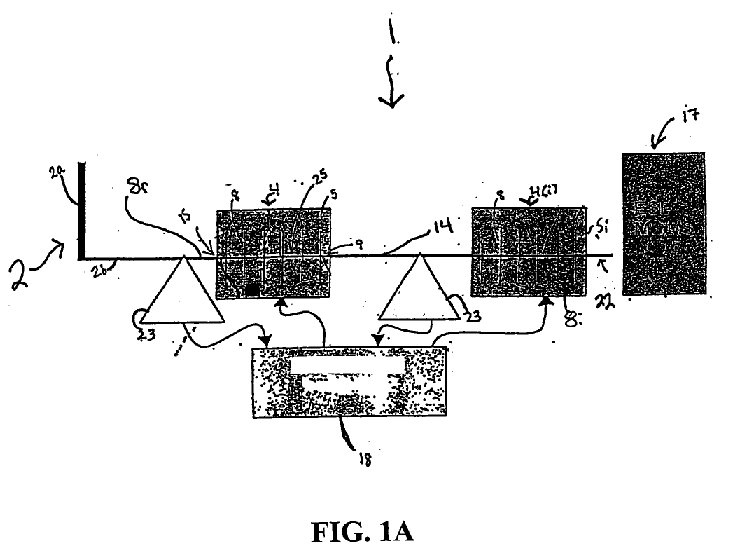

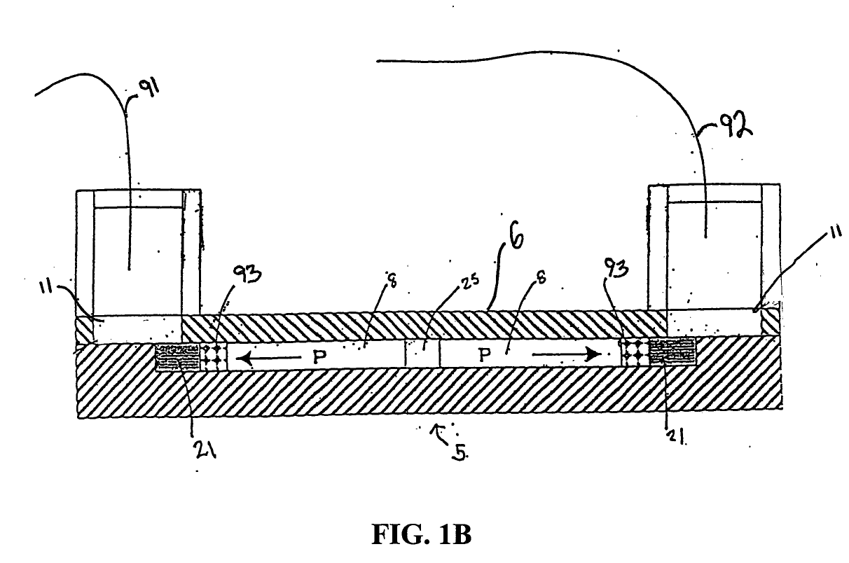

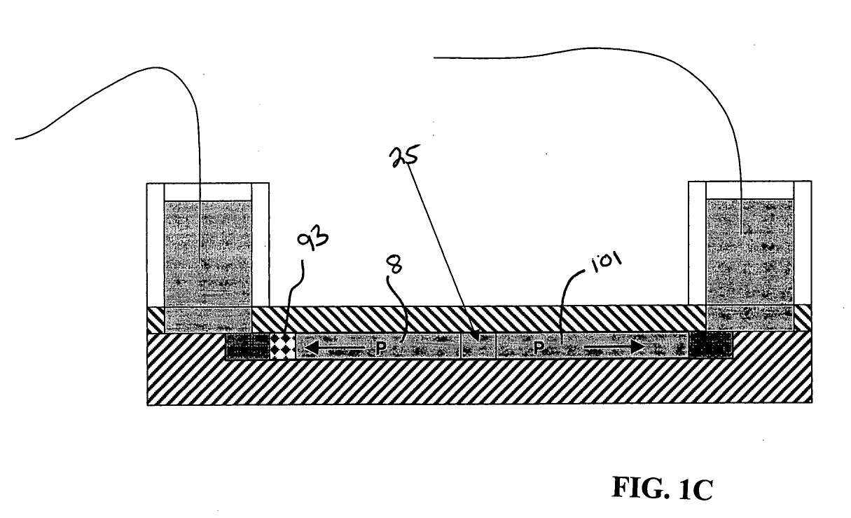

In a preferred aspect (shown in FIG. 1A), an integrated proteomic analysis system 1 comprises an upstream separation module 2, preferably a multi-dimensional chromatography device comprising one or more separation columns or channels (e.g., 2a, 2b, etc.) interfaced with at least one microfluidic module 4. The microfluidic module 4 comprises a microfluidic device 5 which is a substrate comprising one or more recipient channels 8r for receiving substantially purified polypeptides from the upstream separation module 2. Preferably, the microfluidic device 5 is covered by an overlying substrate (e.g., a coverglass, not shown) which comprises openings communicating with the one or more channels of the microfluidic device 5 and through which solutions and/or reagents can be introduced into the channels. The overlying substrate also maintains the microfluidic device 5. as a substantially contained environment, minimizing evaporation of solutions flowing through the channels of the microfluidic device 5.

In a preferred aspect, an enzyme is immobilized in one or more reaction channels 8 of at least one of the microfluidic devices 5 of the system 1 generating an “on-device” protein digestion system. Still more preferably, as polypeptides travel through reaction channels 8 of the microfluidic device 5 by mass transport, they are concentrated as they are digested by the proteases. In one aspect, the microfluidic device 5 is coupled at its downstream end to a downstream separation module 14 (e.g., such as a capillary electrophoresis or CE module) which collects digested polypeptide products, i.e., peptides, and which can perform further separation of these peptides. The downstream separation module 14 is in communication with a peptide analysis module 17 (e.g., an electrospray tandem mass spectrometer or ESI-MS/MS) which is used to collect information relating to the properties of the individual peptides. One or more interfacing microfluidic modules 4i also can be provided for interfacing the downstream separation module 14 with the peptide analysis module 17.

Preferably, the system 1 further comprises a system processor 18 which can convert electrical signals obtained from different modules of the system 1 (and/or from their own associated processors or microprocessors) into information relating to separation efficacy and the properties of substantially separated proteins and peptides as they travel through different modules of the system. Preferably, the system processor 18 also monitors the rates at which proteins/peptides move through different modules of the system. Preferably, signals are obtained from one or more detectors 23 which are in optical communication with different modules and/or channels of the system 1. In one embodiment, the detectors 23 are in communication with the upstream separation module 2 and as such are able to deliver a sample plug to a correct location of the microfluidic device 5 in order to undergo a digestion reaction.

The system 1 can vary in the arrangements and numbers of components/modules within the system. For example, the number and arrangement of detectors 23 can vary. In one aspect, the microfluidic device 5 can interface directly with the peptide analysis module 17 without connection to an intervening downstream separation module 14 and/or interfacing module 4i or can interface to the downstream separation module 14 and not an interfacing module 4i, or to an interfacing module 4i but not a downstream separation module 14, etc. In some aspects, the microfluidic device 5 also can perform separation, eliminating the need for one or more separation functions of the upstream separation module 2. In still other aspects, the interfacing module 4i can be coupled to a separation module for connection to a peptide analysis module 17 without connection to a microfluidic device 5. In this scenario, digested or partially digested polypeptides can be delivered to the separation module after being obtained from an interfacing module 4i not connected to the system 1, or less preferably, after being obtained from an on-gel digestion process.

Further, although the system 1 is described as being “integrated” in the sense that the different modules complement each others' functions, various components of the system can be used separately and/or in conjunction with other systems. For example, components selected from the group consisting of: the upstream separation module 2, microfluidic device 5, downstream separation module 14, interfacing module 4i, and peptide analysis module 17, and combinations thereof, can be used separately. Additionally, some modules can be repeated within the system 1, e.g., there may be more than one upstream and/or downstream separation module (2 and/or 14), more than one microfluidic device, more than one interfacing module 4i, more than one detector 23, and more than one peptide analysis module 17 within the system 1. It should be obvious to those of skill in the art that many permutations are possible and that all of these permutations are encompassed within the scope of the invention.

Upstream Separation Modules

In a preferred aspect of the invention, the upstream separation module 2 comprises a separation of a least one-dimension. In one embodiment, the upstream separation module 2 comprises a capillary electrophoresis device. However, a preferred version would use a multi-dimensional column separation device. Any combination of chemical separation systems that are mutually compatible could be combining, which would include but not be limited to all of the various modes of chromatography, electrophoresis, and diffusion based separations. In multi-dimensional separations, samples are separated in at least two-dimensions in accordance with different criteria. For example, in a first dimension, components in a sample may be separated using isoelectric focusing providing information relating to the isoelectric point of a component of interest and in the second dimension, components having the same isoelectric point can be separated further according to molar mass.

In one aspect, as shown in FIG. 1A, the upstream separation module 2 comprises at least a first and a second separation path, 2a and 2b, respectively. In one aspect, at least one of the separation paths is a capillary. In another aspect, both separation paths are capillaries. The first and second separation paths comprise first and second separation medium.

In one aspect, the first separation path is a capillary coupled to an injection device (e.g., such as a micropipettor, not shown) which injects or delivers a sample comprising a mixture of polypeptides to be separated into the first separation medium. In a preferred aspect, a sample comprises a lysate of cell(s), tissue(s), organism(s) (e.g., microorganisms such as bacteria or yeast) and the like. In a particularly preferred aspect, a sample comprises a lysate of abnormally proliferating cells (e.g., such as cancerous cells from a tumor). Samples also can comprise subcellular fractions such as those which are enriched for particular organelles (e.g., such as nuclei or mitochondria). In one aspect, proteins are concentrated prior to separation. Preferably, the sample which is injected comprises micrograms of polypeptides.

One or more electrodes (not shown) coupled at least at a first and second end of the first separation path 2a is used to create an electric field along the separation path. In one aspect, a second separation path 2b connects to the first separation path, receiving samples from the first separation path 2a which have been substantially separated according to a first criteria. Passage of the separated samples through the second separation path 2b substantially separates these samples according to a second criteria. Multiple parallel separation paths 2b also can be provided for separating samples in parallel. Systems and methods for controlling the flow of samples in separation paths are described in U.S. Pat. No. 5,942,093.

The region of intersection of the first and second separating paths, 2a and 2b, forms an injection device for injecting the sample substantially separated according to the first criteria into the second separation medium. If capillary electrophoresis is used for the separation 2b, an electric field applied along the second separating path 2b then causes the samples substantially separated according to the first criteria to become substantially separated according to the second criteria. In one aspect, one or more waste paths (not shown) are provided to draw off unwanted carrier medium (see, e.g., as described in U.S. Pat. No. 5,599,432).

Additional separation paths can be provided downstream of the first separation path 2a, for example, connected to the second separation path or between the first and second separation path. Each of these additional paths can perform separations using the same or different criteria as upstream separation paths.

In one aspect, at least one separation medium in at least one separation path is used to establish a pH gradient in the path. For example, ampholytes can be used as the first separation medium. The first separation path can be connected at one end to a reservoir portion (not shown) and at other end to a collecting path (not shown) proximate to the intersection point between the first and second path. Electrodes can be used to generate an electric field in a reservoir comprising the ampholyte and in the collecting path. The acidic and basic groups of the molecules of the ampholyte will align themselves accordingly in the electric field, migrate, and in that way generate a temporary or stable pH gradient in the ampholyte.

Different separating paths, reservoirs, collecting paths, and waste paths can be isolated from other paths in the upstream separation module 2 using valves operating in different configurations to either release fluid into a path, remove fluid from a path, or prevent fluid from entering a path (see, e.g., as described in U.S. Pat. No. 5,240,577, the entirety of which is incorporated by reference herein). Controlling voltage differences in various portions of the module 2 also can be used to achieve the same effect. Preferably, the opening or closing of valves or changes in potential is controlled by the processor 18, which is further in communication with one or more detectors 23 which monitors the separation of components in different paths within the module 2 (see, e.g., as described in U.S. Pat. No. 5,240,577).

In this way, the first separating path 2a can be used to perform isoelectric focusing while the second separating path 2b can be used to separate components by another criteria such as by mass. However, it should be obvious to those of skill in the art that isoelectric focusing also could be performed in the second path 2b while separation by mass could be performed in the first path by changing the configuration of the reservoir and collecting path. In still further aspects, multiple different pH gradients can be established in multiple different separation paths in the upstream separation module 2.

The choice of buffers and reagents in the upstream separation module 2 will be optimized to be compatible with a downstream system with which it connects, such as a microfluidic device 5 which can perform protease digestion of separated samples (described further below). Preferably, a buffer is selected which maintains polypeptide/peptide solubility while not substantially affecting reactions occurring in the downstream system (e.g., such as protease digestion and ultimately, protein analysis). For example, acetonitrile (ACN) and solubizing agents such as urea and guanidine can be used as these will not affect analyses such as trypsin digestion (such as would occur in the downstream microfluidic module 5) or ionization (such as would occur in the downstream peptide analysis module 17). Although not required, when a CE column is used as an upstream separation module, a solid-phase extraction (SPE) CE system that incorporates an SPE bead can be provided upstream of the CE column, enabling buffers to be changed and samples to be concentrated prior to CE separation. Commercially available chromatography beads have been designed specifically for the extraction of proteins from detergent containing solutions (Michrom Bioresources, Auburn, Calif.). Elution from the SPE also can achieved with ACN.

In a currently preferred aspect, at least one separation is performed which relies on size-exclusion, e.g., such as size-exclusion chromatography (SEC) (see, e.g., Guillaume, et al., 2001, Anal. Chem. 73(13): 3059-64). Ion-exchange also can be employed and has the advantage of being a gradient technique. Both of these separations are compatible with the surfactants and denaturants used to maintain protein solubility. In another aspect, at least one separation is a chromatofocusing (CF) separation. CF separates on the basis of isoelectric point (pI) and can be used to prepare milligram quantities of proteins (see, e.g., Burness et al., 1983, J Chromatogr. 259(3): 423-32; Gerard et al., 1982, J Immunol. Methods 55(2): 243-51. Preferably, SEC is performed in the first separating path 2a, and CF is performed in the second separating path 2b, achieving a level and quality of separation similar to 2DE.

Parallel separations can be incorporated readily into the system according to the invention, as microfluidic devices comprising up to about 96 channels or more have been fabricated (see, as described in, Simpson et al., 1998, Proc. Nat. Acad. Sci. USA 95: 2256-2261; Liu et al., 1999, Analytical Chemistry 71: 566-573, for example).

However, because the upstream separation module 2 preferably is used to concentrate macrovolumes (i.e., microliters vs. nanoliters) comprising micrograms of sample, it is preferred that at least one component of the upstream separation module be able to concentrate macrovolume samples and separate polypeptides within such sample. In a particularly preferred aspect, therefore, the upstream separation module 2 comprises one or more chromatography columns, preferably, at least one capillary electrochromatography column.

For example, the separation path can comprise a separation medium comprising tightly packed beads, gel, or other appropriate particulate material to provide a large surface area over which a fluid comprising sample components can flow. The large surface area facilitates fluid interactions with the particulate material, and the tightly packed, random spacing of the particulate material forces the liquid to travel over a much longer effective path than the actual length of the separation path. The components of a sample passing through the separation path interact with the stationary phase (the particles in the separation path) as well as the mobile phase (the liquid eluent flowing through the separation path) based on the partition coefficients for each of the components in the fluid. The partition coefficient is a defined as the ratio of the concentration of a component in a stationary phase to the concentration of a component (e.g., a polypeptide or peptide) in a mobile phase. Therefore, components with large partition coefficients migrate more slowly through the column and elute later.

In a preferred aspect, chromatographic separation in the upstream separation module 2 is facilitated by electrophoresis. Preferably, the separation occurs in tubes such as is used in capillary electrochromatography (CEC).

CEC combines the electrically driven flow characteristics of electrophoretic separation methods with the use of solid stationary phases typical of liquid chromatography, although smaller particle sizes are generally used. It couples the separation power of reversed-phase liquid chromatography with the high efficiencies of capillary electrophoresis. Higher efficiencies are obtainable for capillary electrochromatography separations over liquid chromatography. In contrast to electrophoresis, capillary electrochromatography is capable of separating neutral molecules due to analyte partitioning between the stationary and mobile phases of the column particles using a liquid chromatography separation mechanism.

In CEC, the stationary phase can be either particles which are packed into capillary tubes (packed CEC) or can be attached (i.e., modified or coated) onto the walls of the capillary (open tubular or OTEC). The stationary phase material is similar to that used in micro-HPLC. The mobile phase, however, is pumped through the capillary column using an applied electric field to create an electro-osmotic flow, similar to that in CZE, rather than using high pressure mechanical pumps. This results in flat flow profiles which provide high separation efficiencies. Therefore, in a currently preferred embodiment, at least one component of the upstream separation module 2 comprises one or more CEC columns.

CEC systems can also be provided as part of a microchip. See, as described in Jacobson et al., 1994, Anal. Chem. 66: 2369-2373, for example.

Microfluidic Module For Protease Digestion

Microfluidic devices have been developed for rapid analysis of large numbers of samples. Compared to other conventional separation devices, microdevice-based separation devices have higher sample throughput, reduced sample and reagent consumption and reduced chemical waste. The liquid flow rates for microdevice-based separation devices range from approximately 1-300 nanoliters (nL) per minute for most applications.

Microfluidic devices offer new methods for handling nL volume solutions without dilution. Their compact format allows for the massive parallelism required for proteome analysis. Arrays of up to 96 capillaries have been fabricated on devices for high throughput DNA sequencing (Simpson et al., 1998, supra; Liu et al., 1999, supra). Further, on-device electroosmotic pumping of sample through different channels of a device can be achieved simply with arrays of electrodes. Controlling an electrode array is much simpler than controlling an array of high pressure lines and valves. Additionally, the closed system architecture reduces contamination and difficulties caused by evaporation.

In one aspect, the system 1 comprises a microfluidic device 5 downstream of the upstream separation module 2 and in communication with the upstream separation module 2 through a recipient channel interface 15 which can comprise one or more recipient channels 8r for connecting to one or more separating paths of the upstream separation module 2.

Preferably, the microfluidic device 5 comprises a biocompatible substrate such as silicon or glass or polymer and comprises one or more reaction channels 8. Preferably, the device comprises at least about 2, at least about 4, at least about 8, at least about 16, at least about 32, at least about 48, or at least about 96 reaction channels 8. Reaction channels 8 can vary in size and are generally from about 50 μm-200 μm wide (preferably, from about 80 μm-100 μm wide) and from about 5 μm-40 μm deep (preferably from about 10 μm-30 μm deep). The substrate is not necessarily planar and may be represented in a three-dimensional channel network.

In one aspect, a microfluidic device 5 is formed by rapid replica molding against a patterned silicon master. Silicon masters can be formed with photolithographic techniques using photoresists. For example, a standard photolithographic procedure consists of sputter coating a silica device with Cr, spin coating with a photoresist (e.g., such as a nSU8 negative photoresist) exposing the photoresist, and etching channels with HF/NH₄F. Methods for channel etching are known in the art and described in Fan et al., 1994, Anal. Chem. 66, 177-184 and Jacobson et al., 1994, Anal. Chem. 66: 1107-1113, for example. Reactive-ion etching, thermal oxidation, photolithography, ion implantation, metal deposition and other standard semiconductor processing techniques also can be used to fabricate the microfluidic device 5.

The device can be substantially covered with an overlying substrate for maintaining a substantially closed system (e.g., resistant to evaporation and sample contamination) (not shown). The overlying substrate can be substantially the same size as the microfluidic device 5, but at least is substantially large enough to cover the various channels of the microfluidic device 5. In one aspect, the overlying substrate comprises at least one opening for communicating with at least one channel in the microfluidic device 5. The openings can be used to add reagents or fluid to the microfluidic device 5. In another aspect, as shown in FIG. 1B, openings can be used to apply an electric voltage to different channels in communication with the openings.

Suitable materials to form the overlying substrate comprise silicon, glass, plastic or another polymer. In one aspect, the overlying substrate 6 comprises a material which is substantially transmissive of light. The overlying substrate 6 can be bonded or fixed to the microfluidic device 5, such as through anodic bonding, sodium silicate bonding, fusion bonding as is known in the art or by glass bonding when both the microfluidic device 5 and overlying substrate 6 comprise glass (see, e.g., as described in Chiem et al., 2000, Sensors and Actuators B 63: 147-152).

The microfluidic device 5 preferably collects substantially separated proteins from the upstream separation module 2 in a recipient channel 8r and the microfluidic device 5 further comprises at least one reaction channel 8 for reacting a sample with one or more proteases or various agents.

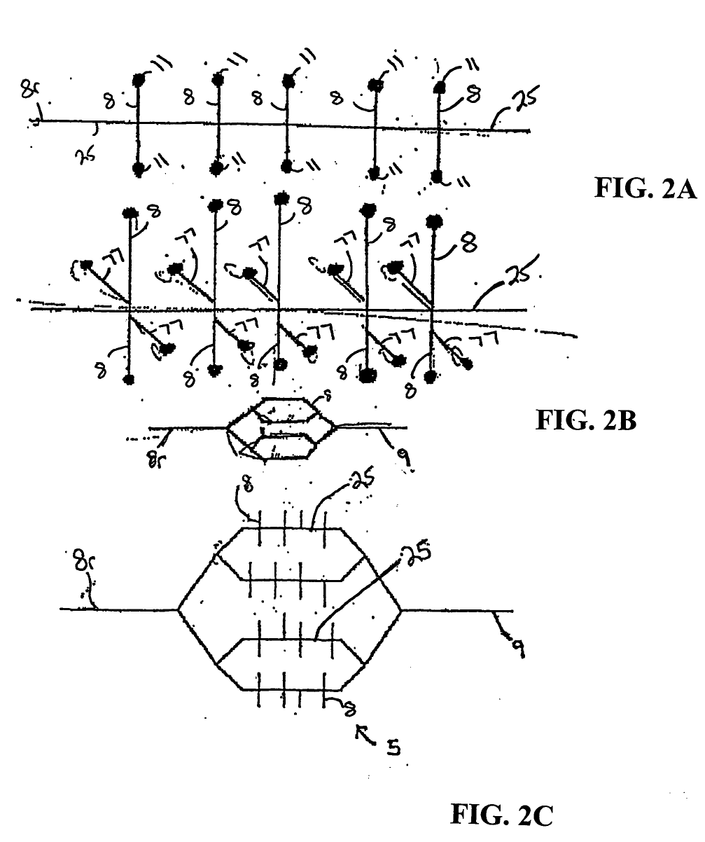

The microfluidic device 5 may comprise varying channel geometries. As shown in FIG. 2A, the recipient channel 8r engages an intersecting channel 25. The intersecting channel 25 travels from the input channel to the output channel. At various positions, the intersecting channel 25 engages reaction channels 8. In one embodiment, the various reaction channels 8 may be in a nearly perpendicular configuration to each other. The intersecting channel delivers a substantially purified polypeptide to a reaction channel 8; once the reaction is complete, a reaction product is returned to the intersecting channel 25 and sent out of the microfluidic device 5. In a preferred aspect of the invention, one or more reservoir channels intersect with the recipient channel 8r and/or reaction channels 8. More preferably, at least one of the reaction channels terminates in a reservoir well 11 that connects with openings in the overlying substrate allowing solutions or reagents to be added to the reservoir 11 of the microfluidic device 5 through the openings. Additionally, auxiliary channels 77 may be incorporated in to the microfluidic device 5 to provide additional reagents as needed.

In FIG. 2B, the microfluidic device 5 comprises a recipient channel which divides into a plurality of substantially parallel reaction channels which converge again at an output channel. FIG. 2C shows another embodiment of the present invention wherein the recipient channel divides into a plurality of intersecting channels 25. The intersecting channels 25 engage a plurality of reaction channels 8. Such configurations allow for high throughput. The absolute channel geometry is not critical so long as the appropriate fluid flow relationships are maintained. For example, channels can be curved and in one aspect, the substrate itself is not planar and the channels can be non-coplanar (e.g., radiating from a central intersection channel as spokes from a central hub).

Many refinements to the geometry of the channel layout can be made to increase the performance of the microfluidic device 5 and such refinements are encompassed within the scope of the invention. For example, shorter channels will decrease the distance over which sample bands must be transported, but generally channels need to be long enough to hold the sample bands, and to provide adequate separation between electrodes in contact with channels (discussed further below) to prevent current feedback.

In addition to the reaction channels 8 for protease digestion, additional channels can be provided. As shown in FIG. 1B, in one aspect, one or more channels are provided which are protease resistant channels 101 (e.g., the channel can comprise one or more protease inhibitors) for moving a sample comprising a substantially purified polypeptide directly to the peptide analysis module 17 to obtain a determination of its mass (e.g., for comparison with digested forms of the polypeptide). In another aspect, one or more reaction channels 8 are provided which comprise derivatizing enzymes and/or chemicals for chemically modifying polypeptides or their digestion products to facilitate the peptide analysis process. In one embodiment, these enzymes are provided through the auxiliary channels 77. In a further aspect, one or more reaction channels 8 can be provided comprising buffers and/or other reagents which can be selectively added to the different other channels of the system. For example, suitable ions can be provided through such channels to change the pH of one or more other channels of the system.

In one embodiment of the present invention, a substantially purified polypeptide may be divided so that a first portion of the substantially purified polypeptide is subjected to a particular enzyme or derivatization and a second portion of the substantially purified polypeptide is not subjected to the particular enzyme or derivatization. In one aspect of the present invention, the first portion of the substantially purified polypeptide is subjected to a particular enzyme and the second portion of the substantially purified polypeptide is sent directly to a peptide analysis module 17 of the present invention. In one aspect of the present invention, the substantially purified polypeptide is divided into a plurality of samples. In one embodiment of the present invention, an at least one sample of the plurality of samples is subjected to a first enzyme and an at least second sample of the plurality of samples is subjected to a second enzyme.

In one embodiment of the present invention, a substantially purified polypeptide is divided into a first sample and a second sample. In one embodiment of the present invention, the first sample is treated with a particular enzyme or derivatizing agent and the second sample is sent directly to a peptide analysis module 17 to be analyzed. In one embodiment of the present invention, the particular enzyme is a phosphatase. In one embodiment of the present invention, after the sample of the substantially purified polypeptide which has been treated with the phosphatase, the sample is sent to the peptide analysis module 17. After the sample which has been treated with the phosphatase is analyzed by the peptide analysis module 17, the data obtained by the peptide analysis module 17 is compared to the dated obtained by the peptide analysis module when analyzing the sample which was not treated with the phosphatase. By analyzing both the sample which has been treated with the phosphatase and the sample which was not treated by the phosphatase and comparing the resulting data, a greater degree of information may be obtained regarding the substantially purified polypeptide.

In one embodiment of the present invention, the first sample of the substantially purified polypeptide is treated by a cross-linking enzyme and the second sample is not treated by a cross-linking enzyme. In one aspect of the present invention, the second sample is sent directly to the peptide analysis module 17 of the present invention. In one embodiment of the present invention, the three-dimensional structure and/or spatial orientation of a protein, peptide, or polypeptide by crosslinking the protein prior to digestion on the microfluidic chip and analyzing the crosslinked fragments, which maintain the spatial orientation of the original peptide. Those of skill in the art will recognize that many enzymes and/or derivatizing agents are within the spirit and scope of the present invention.

Preferably, the microfluidic module 4 provides a compartment in the system 1 for on-line protein digestion of substantially separated proteins. In one aspect, the microfluidic device 5 comprises proteases immobilized in one or more reaction channels 8 of the device. In contrast, to in-gel digests with proteases, such as trypsin, which can require from about 6 to about 24 hours, “on-device” digests using the microfluidic devices 5 according to the invention can be performed on timescales of minutes with little chemical background. The immobilized protease allows the use of high concentrations of enzyme with negligible production of autolysis products. In contrast, with in-gel digests, the enzyme must permeate the gel, precluding immobilization of the enzyme and resulting in significant autolysis peaks.

In a preferred aspect, proteases are contained within one or more of the reaction channels 8 of the microfluidic device 5. Suitable proteases include, but are not limited to: peptidases, such as aminopeptidases, carboxypeptidases, and endopeptidases (e.g., trypsin, chymotrypsin, thermolysin, endoproteinase Lys C, endoproteinase GluC, endoproteinase ArgC, endoproteinase AspN). Aminopeptidases and carboxypeptidases are useful in characterizing post-translational modifications and processing events. Combinations of proteases also can be used. Where the system comprises a plurality of reaction channels 8, at least one channel can be free of proteases and/or resistant to protease digestion (e.g., can comprise one or more protease inhibitors as described above). Further, different channels can comprise different types or amounts of protease or other enzymes or derivatizing chemicals to perform a plurality of reactions of substantially identical samples (e.g., obtained from a single sample plug) in parallel. Agents for sequence-specific cleavage also can be provided such as, and the like.

Further, the extent of digestion may be controlled by precisely controlling the amount of time a sample is exposed to protease to produce larger peptides or peptides comprising overlapping sequences. Moreover, a portion of a polypeptide sample can be excluded from proteolytic digestion in order to measure the molecular mass of the intact polypeptide.

In one aspect, proteases are immobilized on a first solid phase, such as particles, within the one or more reaction channels 8. Particle materials useful for the invention include, but are not limited to: silica, glass, polystyrene, or other polymeric compositions such as agarose or sepharose. Chromatographic beads 93 (e.g., Spherisorb ODS1 beads, available from Phase Separations, Flintshire, UK), and porous C-18 beads also can be used. Immobilized trypsin beads are commercially available. Particles can vary in size depending on the channel diameters of the device and in one aspect, can range from 1.5-4.0 μm in diameter. Preferably, the particles themselves are substantially immobilized in the reaction channels 8.

Preferably, bead injection technology is used to add or replace the beads 93 as is known in the art (see, e.g., Ruzicka and Scampavia, 1999 Anal. Chem. 71(7): 257A-263A; Oleschuk et al., 2000, Anal. Chem. 72(3): 585-590).

While capillary systems for performing proteolytic digestions (see, e.g., Licklider et al., 1995, Analytical Chemistry 67: 4170-4177; Licklider et al., 1998, Analytical Chemistry 70: 1902-1908) and microfluidic devices for protease digestion have been described (see, e.g., Tremblay et al., 2001, Proteomics 1(8): 975-986; Li et al., 2001, Eur. J Mass Spectrom. 7(2): 143-155; Li et al., 1999, Anal. Chem. 71: 3036-3045; Khandurina et al., Anal. Chem. 71: 1815-1819), these devices have not concentrated samples during digestion and have not been used in a format to selectively collect samples from an upstream separation module. In contrast to prior art systems, the present system makes digestion kinetics more favorable for dilute samples.

In further aspects, at least a portion of a reaction channel 8 of the microfluidic device 5 comprises one or more enzymes which can add chemical moieties to a protein or peptide or remove chemical moieties from a protein or peptide to facilitate further downstream separation or analysis.

Proteases and/or other enzymes can be immobilized onto particles using adsorptive or covalent methods. Covalently immobilized enzymes are generally preferred because the enzymes remain immobilized longer and are more stable under a wide variety of conditions. Common examples of covalent immobilization include direct covalent attachment of the protease to an alkylamine-activated particle with ligands such as glutaraldehyde, isothiocyanate, and cyanogen bromide. However, proteases also can be immobilized on a solid phase using binding partners which specifically react with the proteases or which bind to or react with molecules which are themselves coupled to the proteases (e.g., covalently). Binding partners preferably have affinity constants greater than about 10⁸ or a dissociation constant of about 10⁻⁸. Representative examples of suitable ligand binding pairs include cytostatin/papain, valphosphanate/carboxypeptidase A, biotin/streptavidin, riboflavin/riboflavin binding protein, and antigen/antibody binding pairs.

Preferably, the binding pair or molecule bound to the binding pair is positioned away from the catalytic site of the protease and/or other enzyme.

Particles comprising proteases and/or other enzymes can be packed into the reaction channels 8 of the device by applying voltages at selected channels to drive the particles into the desired channels. Preferably, the particles comprise charged surface molecules (e.g., such as free silonol groups) to facilitate this process. For example, electroosmotic flow driven by walls of the reaction channels 8 and free silonol groups on the particles can be used to effect packing. In one aspect, a voltage of from about 200-800 V for about 5 minutes at a selected reaction channel 8 while remaining, non-selected channels are grounded, is sufficient to drive particles into the selected reaction channel 8. Packing of particles also may be performed electrokinetically as described in U.S. Pat. No. 5,942,093.

However, in another aspect, particles are magnetic, paramagnetic or superparamagnetic, and can be added to or removed from the reaction channels 8 of the microfluidic device 5 by using a magnetic field applied to selective regions of the microfluidic device 5.

Initially, particles can be delivered into the reaction channels 8 in a solvent such as (acetonitrile) ACN. Trypsin has a high tolerance to ACN, and is actually efficient at about 10% ACN, with reports of up to about 40% ACN (see, e.g., Figeys et al., 1998, Electrophoresis 19: 2338-2347), and 80% having been used effectively. As discussed above, these conditions also are compatible with buffers used in upstream separation modules, such as CEC devices.

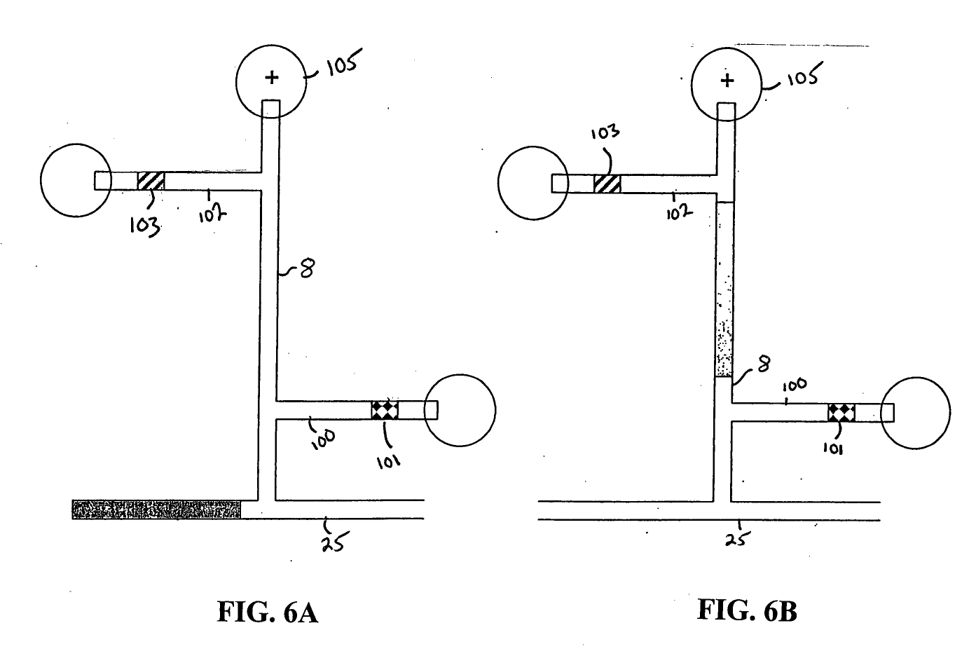

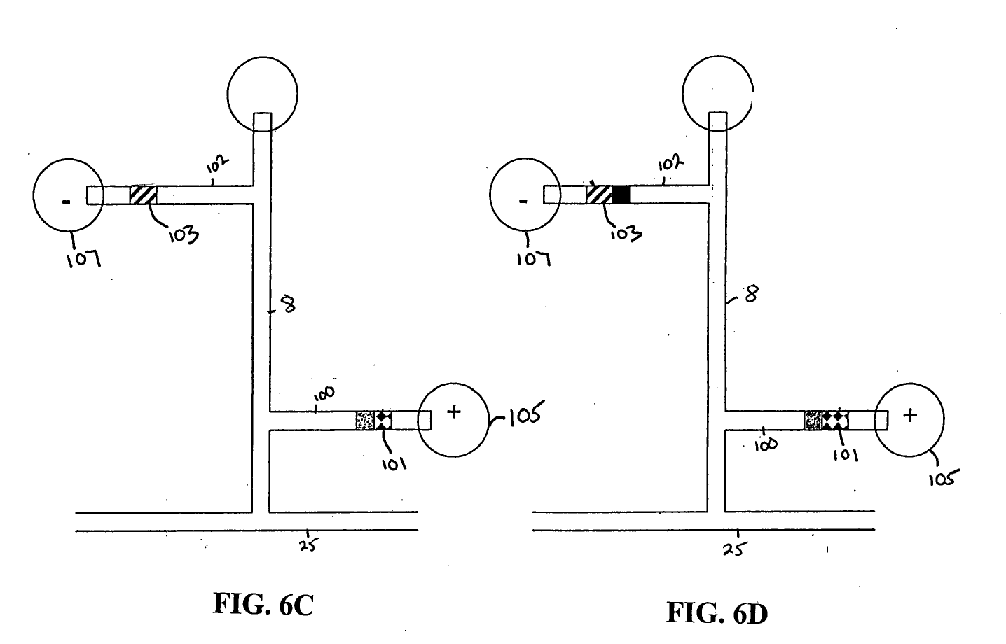

In a currently preferred aspect, as shown in FIG. 1B, where one or more reaction channels are provided which intersect with an intersecting channel 25, at least a portion of the reaction channel 8 comprises a second solid phase (e.g., a sol-gel membrane, filter, membrane, or frit) 21 through which a current can move but not polypeptides or digestion products of the polypeptides. Because polypeptides are concentrated as they are digested, low concentration samples can be digested more quickly with fewer autolysis products. Preferably, a second solid phase (to be discussed with FIGS. 6A-8D) is a nanocapillary array (or a nanochannel array). In the presence of an electric field, to be discussed below, the molecular weight cut-offs are different from the molecular weight cut-offs in a typical ultrafiltration driven by hydrodynamic flow.

In one embodiment of the present invention, a reaction channel 8 of the microfluidic device 5 does not comprise a first solid phase 93. In one embodiment, the protease remains in a liquid phase (and therefore does not require the first solid phase 93). In FIG. 1B, the variable “P” represents a change in pressure wherein the pressure change forces the substantially purified polypeptides to move in a desired direction.

In devices which have the substantially parallel channel configuration shown in FIG. 2B, concentration preferably is achieved by holding samples in the channels and focusing them, e.g., by creating a pH gradient in the channels as described further below.

In a preferred embodiment of the present invention, the system is driven by electroosmotic flow. Preferably, the microfluidic device 5 comprises at least one electrode in communication with one or more channels in the microfluidic device 5 to drive mass transport of polypeptides through the various channels of the microfluidic device 5. In a preferred aspect, flow of solution comprising polypeptides is controlled electroosmotically and electrophoretically by control of voltage through the electrode(s). In one aspect, providing a silicon oxide layer on a surface of the microfluidic device 5 provides a surface on which conductive electrodes can be formed (e.g., by chemical vapor deposition, photolithography, and the like). The thickness of the layer can be controlled through oxidation temperature and time and the final thickness can be selected to provide the desired degree of electrical isolation. In a preferred aspect, a layer of silicon oxide is provided which is thick enough to isolate electrode(s) from the overlying substrate thereby allowing for the selective application of electric potential differences between spatially separated locations in the different channels of the device 5, resulting in control of the fluid flow through the different channels. In aspects where the overlying substrate is not glass, one or more electrodes also can be formed on the overlying substrate.

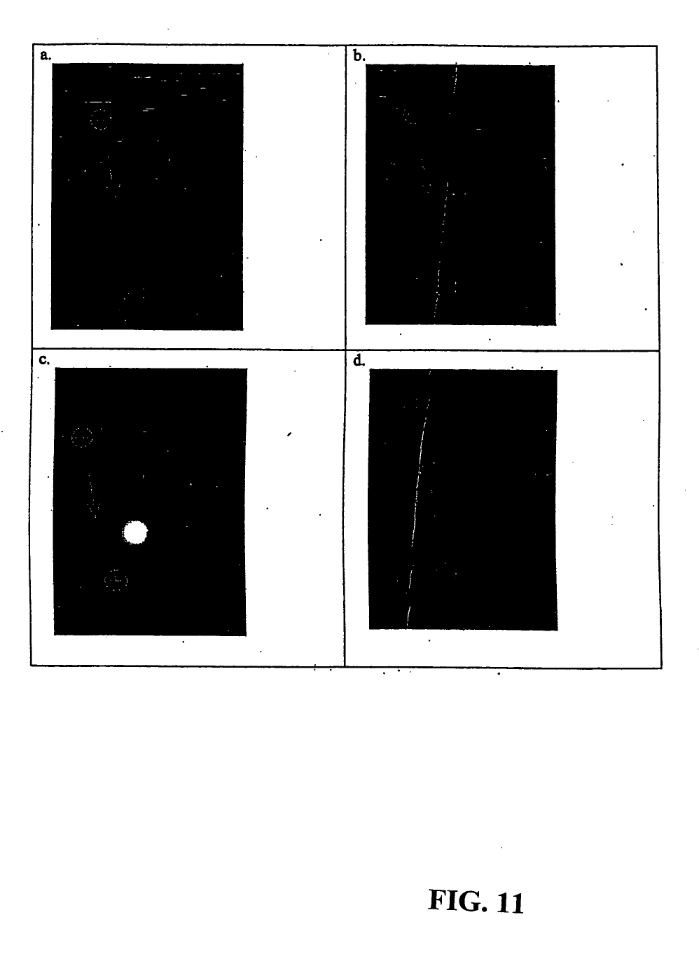

In another aspect of the present invention, as shown in FIG. 11B and FIG. 1C, one or more electrodes can be in electrical communication with a buffer solution provided in a reservoir well 11 at the terminal end of a reaction channel 8.

In one aspect of the present invention, however, flow through one or more selected channels of the device is hydrodynamic and mediated mechanically through valves placed at appropriate channel junctions as is known in the art. See, e.g., as described in U.S. Pat. No. 6,136,212; U.S. Pat. No. 6,008,893, and Smits, Sensors and Actuators A21-A23: 203 (1990). To improve sample handling and ultimately improve detection limits of the system precise control of flow is required. Therefore, in one aspect, flow of reagents in each of the channels of the device 5 is independently controlled. Preferably, transport is voltage driven rather than pressure driven. To prevent or reduce feedback or cross talk between channels, electrodes and buffer reservoirs along undesired alternative paths can be used to block feedback by acting as current and electroosmotic flow drains.

To prevent feedback through connected channels, a series of electrodes can be used that act as either a source or drain of electroosmotic flow. If high currents are passed through the drains, problems can arise from Joule heating or rapid consumption of buffer. Buffer consumption is a technical problem that can be solved by appropriate engineering (e.g., providing reservoirs 11 through which buffers can be added). Buffer out-gassing, which can occur at high levels of Joule heating can be avoided by degassing buffers before use. The maximum voltage used is largely governed by out-gassing of the buffer solutions used in the system. Since current is proportional to voltage, at higher voltages there will be more Joule heating and a greater tendency for out-gassing to occur. With the current scheme of voltage control for sample transport the largest current will flow between the electrodes that are acting as potential and electroosmotic flow sinks, and these are the areas where outgassing will be most likely. However, very high electric field strengths can be used with microdevices as ultrafast separations have been carried out at 53 kV/cm (see, e.g., Figeys et al., 1997, J Chromatogr., 763: 295-306) and the present invention contemplates the use of high voltage for rapid sample transport, but an electric field strength below 53 kV/cm.

The microfluidic device 5 collects sample bands comprising substantially purified polypeptides as they elute from an upstream separation module 2 as shown in FIG. 1A. Preferably, an optical detector 23 located near the recipient channel interface 15 will detect the separated sample bands. The rate at which bands reach this optical detector 23 will be used to compute the mobility of the bands and the time at which the electrode voltage should be modulated on the microfluidic device to direct the flow of sample. In such a manner, the detector 23 may direct the sample plug to an appropriate reaction channel 8 on the microfluidic device 5. When the upstream separation module 2 comprises a CEC device, electroosmotic flow from the upstream separation module 2 can be measured, rather than velocity.

Fluid can be directed into one or more reservoirs 11 above the device if necessary, so only polypeptide bands are sent to the reaction channels 8. Preferably, any running buffer from the upstream separation module 2 between sample peaks that does not contain any sample will be eliminated so it does not take up any space within the microfluidic module 4. Elimination of buffer decreases the amount of time the downstream peptide analysis module will spend analyzing a sample without peptides, thereby increasing the efficiency of the system 1.

Modulation of the potential at the appropriate electrodes in the array will direct the sample band to the proper channel. Once the protein sample band is held in one of the parallel buffer channels it can be digested by immobilized enzyme within the channel.

The production of bubbles at electrodes can be problematic. Bubbles will be physically separated from the channels when electrodes are held in the buffer reservoirs above the device (see, e.g., as shown in FIG. 1B and FIG. 1C) and where the solution in the reservoirs is connected directly with a channel through a hole in the overlying substrate. If the electrodes are integrated directly onto the channels, then buffer additives can be used to suppress bubble formation, as previously reported for an electrospray MS interface (see, e.g., as described in Moini et al., 1999, Analytical Chemistry 71: 1658-1661).

Where reaction channels 8 are in the substantially parallel configuration as shown in FIG. 2B, for example, electroosmotic pressure induced in the reaction channels 8 through intersection with adjacent channels 8 may slowly force sample bands out and decrease the efficiency of the protease digestion process. By providing an on-device imaging detector 23 (discussed further below) in optical communication with one or more of the reaction channels 8, a user can determine whether sample bands comprising polypeptides and/or their digestion products are actually stationary. If they are not stationary, many different methods can be used to counter the effects of this pressure. For example, electroosmotic flow can be actively controlled by controlling the double layer potential as described by Lee et al., 1990, Anal. Chem. 62: 1550-1552; Wu et al., 1992, Anal. Chem. 64: 886-891; Hayes et al., 1993, Anal. Chem. 65: 27-31; Hayes et al., 1993, Anal. Chem. 65: 2010-2013; and Hayes et al., 1992, Anal. Chem. 64: 512-516. Fabrication of a microfabricated device with such control was recently demonstrated by Schasfoort et al., 1999, Science 286: 942-945.

Electroosmotic pressure in channels having a substantially parallel channel configuration also can be stopped by temporarily breaking electrical contact in the channel. Here, bubbles are desirable and are introduced by low pressure into channel(s) 8 to manipulate flow on the microfluidic device 5. Bubbles can be introduced by physically separating sample plugs or by breaking the electrical conductivity in the channel(s). Strategic positioning of a membrane (e.g., such as a hydrophobic membrane made from polypropylene, polyethylene, polyurethane, polymethylpentene, polytetrafluoroethylene, and the like) which is permeable to the bubbles but not the liquid also can be used for bubble removal. By allowing gas to pass through, but not solution, such a membrane can used to direct solution flow. Gas permeable membranes are known in the art and are described in U.S. Pat. No. 6,267,926, for example. In a similar manner, a hydrophobic coating strategically located after a channel intersection can be used for fabrication of on-device passive valves. See, e.g., as described in McNeely et al., 1999, SPIE: Bellingham 3877: 210-220.

The microfluidic device 5 can be optimized to provide the minimum number of electrode controls per device 5, for example, by tying some of the electrodes together. Incorporation of voltage dividers into the circuitry which is part of the microfluidic device 5 can be used to always hold a pair of electrodes at the same relative potential, while their absolute potentials are varied. Such schemes would reduce the number of high voltage power supplies and control channels required by a processor in communication with the device 5.

The above discussion illustrated the geometric configuration of the present invention; further, the above disclosed the preferred geometric configuration to generate electroosmotic flow for the present invention. A major advantage of the present invention is the ability to concentrate analytes as they are produced in the reaction channels 8 of a microfluidic device 5 driven by electroosmotic flow.

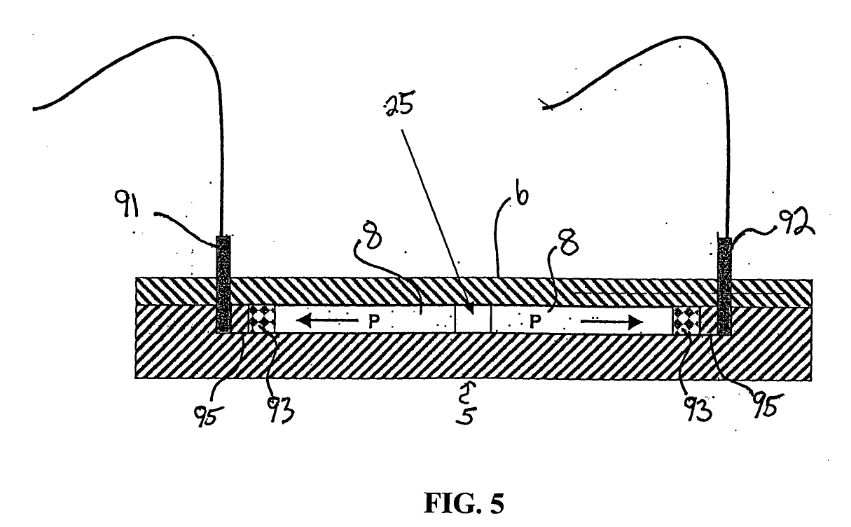

FIG. 5 shows an embodiment of the invention wherein a first electrode 91 is engaged at a first end of a reaction channel 8 and a second electrode 92 is engaged at a second end of the reaction channel 8. An embodiment of the present invention comprises an enzyme immobilized on a plurality of beads 93. An embodiment of the present invention provides a coating 95 layer adjacent to the first electrode 91 and the second electrode 92. In FIG. 5, the variable “P” represents a change in pressure wherein the pressure change forces the substantially purified polypeptides to move in a desired direction.

Current research has demonstrated that bulk flow of solution (provided by electroosmotic flow in these microfluidic systems) is greatly decreased by the integration of the membrane into the channel. Additionally, when concentrating analytes following digestion, a charge trapping effect has been observed which means we can trap small molecules (such as peptides) using large pore membranes. However, the charge trapping is dependent on the analyte charge and the surface charge of the membrane, and cations and anions can not be concentrated together. Also, coatings to reduce loss of proteins on the channel walls typically decreases the charge of the surface and therefore decreases the electroosmotic flow. On the other hand, EOF in the intersecting channel is advantageous for coupling to an upstream separation such as HPLC to provide a bulk flow of solution.

In accordance with the above-mentioned findings, the present invention provides for the design of side channels that incorporate both positively and negatively charged membranes. Since there are advantages to both high EOF system and low EOF systems, the present invention provides a device and method capable of utilizing both a high EOF system and a low EOF system. The present invention for low EOF can be used for both cationic and anionic proteins.

FIGS. 6A, 6B, 6C and 6D shows an embodiment of the present invention in which the reaction channel 8 comprises a first side channel 100 and a second side channel 102. In FIGS. 6A, 6B, 6C, and 6D, a circle represents a series of outlet reservoirs that are above the channels. The channels and the reservoirs are in fluid and electrical communication with each other. The reservoirs contain the voltage control electrodes and the sign of the voltage is shown only when a voltage is applied. The first side channel 100 comprises a negatively charged membrane 101 and the second side channel 102 comprises a positively charged membrane 103. FIG. 6A shows an anionic analyte (protein) in the intersecting channel 25 of a microfluidic device 5. FIG. 6A further comprises a positive electrode 105. FIG. 6B shows the anionic analyte leaving the intersecting channel 25 and traveling toward the positive electrode 105. In FIG. 6B, the potential at the end of the electrode used to effect mass transport down the reaction channel 8 and because there is no membrane the electroosmotic flow is high during steps one (corresponding to FIG. 6A) and two (corresponding to FIG. 6B). FIGS. 6C and 6D show the activation of a positive electrode 105 adjacent to the negatively charged membrane 101 and the activation of a negative electrode 107 adjacent to the positively charged membrane 103. FIG. 6D shows that negatively charged peptides are concentrated at the negatively charged membrane 101 during and after digestion and positively charged peptides are concentrated at the positively charged membrane 103 during and after digestion. The top reservoir can be used to provide an appropriate buffer/reactants for digestion. After the digestion, the concentrated samples can be removed from their corresponding membrane 101,103 and returned to the intersecting channel 25 by adjusting the position of electrodes.

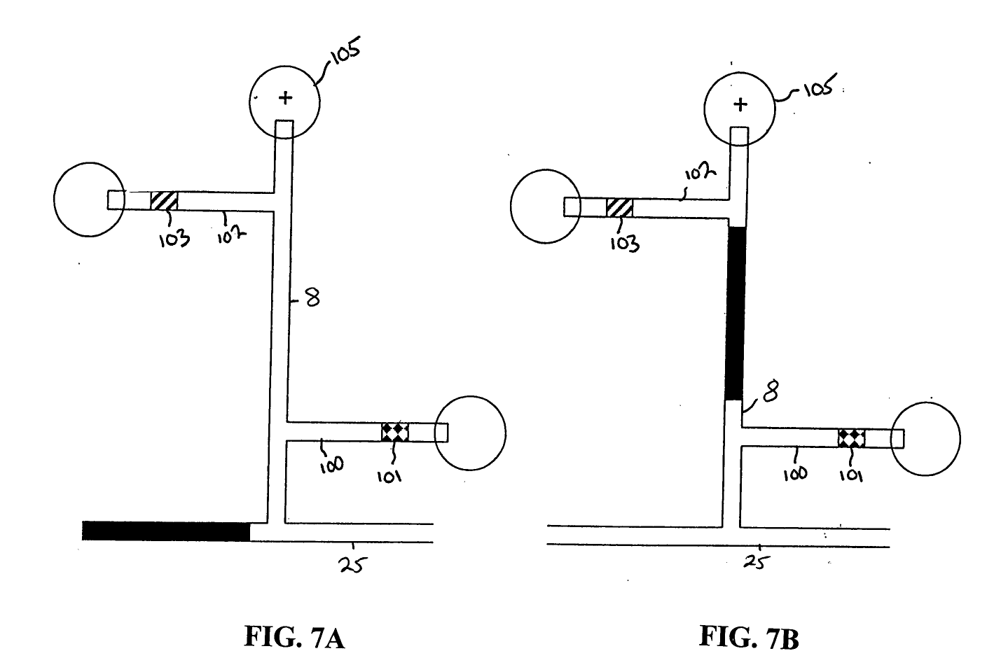

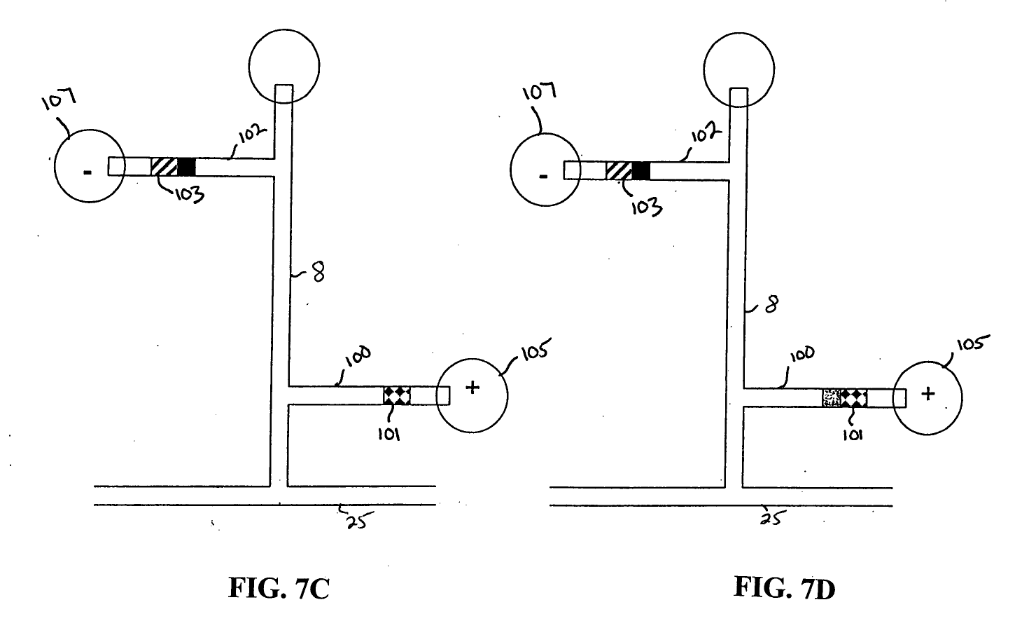

FIGS. 7A, 7B, 7C and 7D shows an embodiment of the present invention in which the microfluidic module 5 comprises a first side channel 100 and a second side channel 102. In FIGS. 7A, 7B, 7C, and 7D, a circle represents a series of outlet reservoirs that are above the channels. The channels and the reservoirs are in fluid and electrical communication with each other. The reservoirs contain the voltage control electrodes and the sign of the voltage is shown only when a voltage is applied. The first side channel 100 comprises a negatively charged membrane 101 and the second side channel 102 comprises a positively charged membrane 103. FIG. 7A shows a cationic analyte (protein) in the intersecting channel 25 of a microfluidic device 5. FIG. 7A further comprises a positive electrode 105. FIG. 7B shows the cationic analyte leaving the intersecting channel 25 and traveling toward the positive electrode 105. In FIG. 7B, the potential at the end of the electrode used to effect mass transport down the reaction channel 8 and because there is no membrane the electroosmotic flow is high during steps one (corresponding to FIG. 7A) and two (corresponding to FIG. 7B). FIGS. 7C and 7D show the activation of a positive electrode 105 adjacent to the negatively charged membrane 101 and the activation of a negative electrode 107 adjacent to the positively charged membrane 103. FIG. 7D shows that negatively charged peptides are concentrated at the negatively charged membrane 101 during and after digestion and positively charged peptides are concentrated at the positively charged membrane 103 during and after digestion. The top reservoir can be used to provide an appropriate buffer/reactants for digestion. After the digestion, the concentrated samples can be removed from their corresponding membrane 101,103 and returned to the intersecting channel 25 by adjusting the position of electrodes.

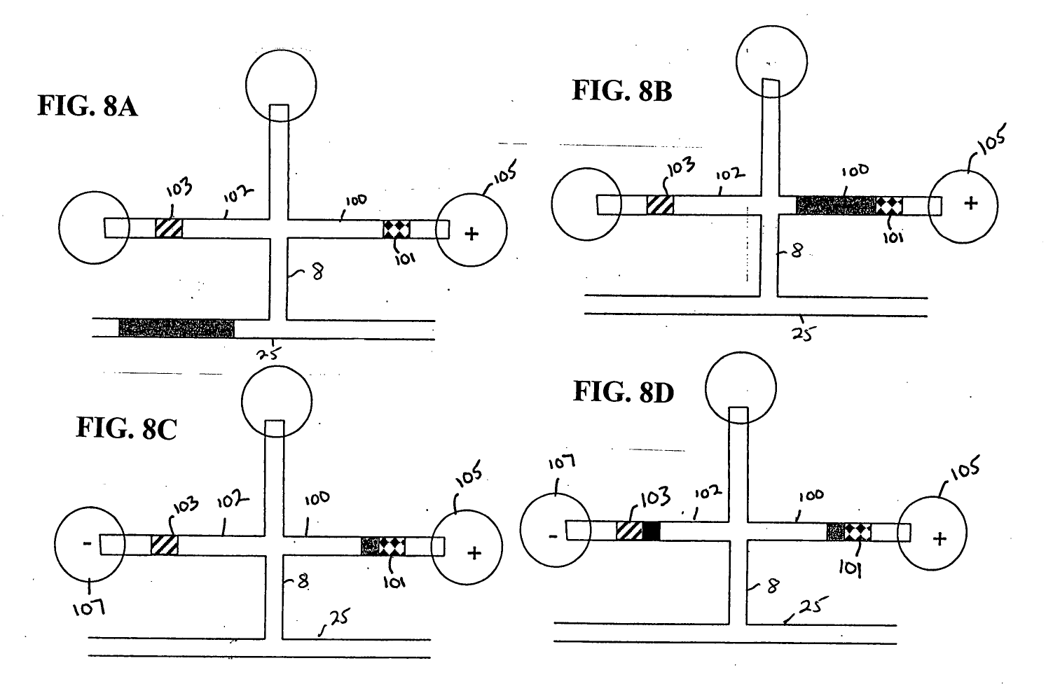

FIGS. 8A, 8B, 8C, and 8D show an embodiment of the present invention in which the intersecting channel 25 has been coated for low electroosmotic flow. In one embodiment of the present invention, the anionic and cationic analytes are separated before entering the intersecting channel 25. FIGS. 8A, 8B, 8C, and 8D comprise a first side channel 100 and a second side channel 102. The first side channel 100 comprises a negatively charged membrane 101 and the second side channel 102 comprises a positively charged membrane 103. FIG. 8A shows an anionic analyte located in the intersecting channel 25 of the microfluidic module 5. FIG. 8A also shows a positive electrode 105 positioned adjacent to the negatively membrane 101. FIG. 8B shows the anionic analyte concentrated in front of the negatively charged membrane 101. FIGS. 8C and 8D show a negative electrode 107 adjacent to the positively charged membrane 103. Further, FIG. 8D shows the negatively charged peptides concentrated in front of the negatively charged membrane 101 before and after digestion and the positively charged peptides concentrated in front of the positively charged membrane 103 before and after digestion. The top reservoir can be used to provide an appropriate buffer/reactants for digestion. After the digestion, the concentrated samples can be removed from their corresponding membrane 101,103 and returned to the intersecting channel 25 by adjusting the position of electrodes.

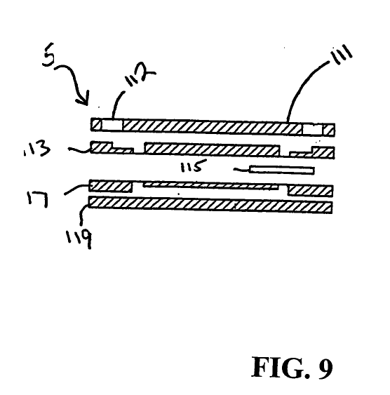

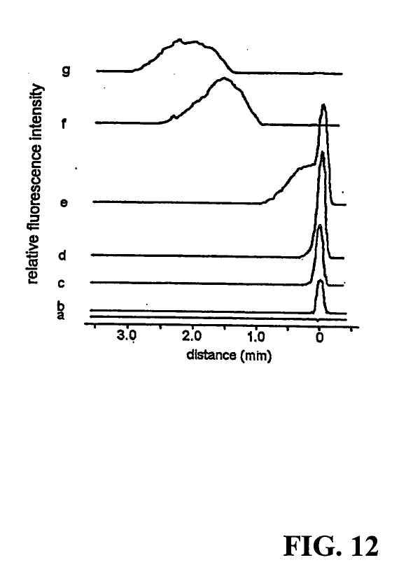

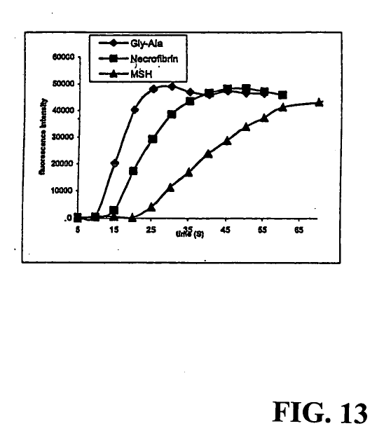

FIGS. 6-8 illustrate the present inventions ability to concentrate analytes. In designing and testing the present invention, an ultrafiltration membrane was integrated into a PDMS microfluidic device for analyte band concentration. Mass transport on the chip was controlled by application of an electric field, and the current was passed directly through the ultrafiltration membrane. The buffer, 50 mM borate, and the electric field strength, 130 V/cm, were similar to those used in capillary electrophoresis and microfluidic devices. 130 V/cm was the maximum field strength that could be used reliably, greater field strengths resulted in unstable current and often lead to current breakdown, presumably caused by bubble formation in the membrane. A 3-D channel structure was fabricated to ensure proper sealing of the device in the microfluidic channel as shown in FIG. 9. The concentration of a 0.1 mM fluorescein solution with a 10 nm PC membrane is shown in FIG. 11. Initially, the fluorescein solution was too dilute to be detached by the CCD imaging system, but as the concentration progressed an intensely fluorescent plug of fluorescein was formed in front of the membrane. Upon reversal of the applied potential a concentrated band of analyte was eluted from the channel. Fluorescence intensity profiles of the same concentration and elution process are shown in FIG. 12. It should be noted that the chips are imaged from above so the volume of solution in the field view is different for the horizontal and vertical sections of the channel. The height of the horizontal channel is about 40 cm while the height of the vertical channel is about 100 μm (measured from the bottom of the horizontal channel to the membrane interface), and the width of the horizontal channel is 100 μm while the diameter of the vertical channel is 150 μm. Thus the distance shown by the x-axis is not directly equal to plug length and the band broadening that occurs upon migration of the plug away from the membrane after reversal of the potential is less than what is directly shown by FIG. 13.

A concentration of factor of 300-fold was measured for the concentration of 1 mM and 0.1 mM fluorescein. Therefore, the maximum obtainable concentration factor is dependent on the initial concentration of the dye. This observation indicates that charge repulsive interactions of fluorescein molecules with each other do not limit the maximum obtainable concentration factor. If charge repulsive interactions of the fluorescein molecules with each other limited the concentration, it is expected that for continuous injections the maximum achievable concentrations of fluorescein would be the same irregardless of the initial concentration. Thus, solutions with different concentrations of fluorescein would yield the same maximum fluorescence (provided all other conditions were held constant). A possibility is that the buffer ions are also concentrated with analyte and limit the maximum obtainable concentration factor. To investigate this possibility 0.1M fluorescein was concentrated in 17, 50 and 100 mM borate buffers. The fluorescence intensity versus time plots were similar, but not identical for all three concentrations.

Studies of analyte concentration were performed using fluorescein and R6G in 50 mM borate buffer at a pH of 8. At this pH, fluorescein is negatively charged while R6G is positively charged, making the pair of dyes useful for investigation of the effects of analyte charge on retention by the membrane. The results indicate that the analyte charge plays a pivotal role in the retention of analytes. Fluorescein was retained by both the 10-nm and 50-nm pore membranes while the R6G was not retained by either. Membranes with both 10-nm and 50-nm pore sizes were used. An important observation is that fluorescein (330 g/mole) was concentrated in front of both the 10-nm and the 50-nm pore membranes although these pore diameters are very large with respect to the size of the molecules. Typically, these ultrafiltration membranes are used for separations driven by hydrodynamic flow through the membrane, and the molecular weight cut-offs for hydrodynamic flow are about 1×10⁵ for the 10-nm pore membrane and 8×10⁵ for the 50-nm pore membrane. Retention and concentration of fluorescein under these conditions demonstrates that the concentration of the dye in front of the membrane and its mass transport across the membrane in electrically driven systems are governed by different mechanisms than those that govern transport in a hydrodynamically driven system. As previously stated, the charge of the molecule plays a dominate role in the concentration as fluorescein could be concentrated using both 10-nm and 50-nm pores, while R6G could not be concentrated by either membrane.

Integration of the membrane into the channel decreased the measured EOF through the channel. The effect of the pore size on the EOF was measured for membranes with 10 nm, 50 nm, and 100 nm. The EOF decreases as the pore size decreases. The measure EOF values are very small and in most cases the contribution of EOF to the overall mass transport the contribution of EOF to the overall mass transport is negligible. Therefore, the analytes are moved toward the membrane by electrokinetic transport, making it impossible to move both anions and cations toward the same membrane simultaneously. For the concentration experiments with R6G the polarity of the electrodes was opposite of the polarity used for fluoroscein.

Ideally, both anions and cations could be concentrated in front of the same membrane simultaneously. Concentration of both anions and cations simultaneously requires the EOF to exceed the magnitude of the electrophoretic mobilities, and a membrane with small enough pores to concentrate based on size. However, obtaining high EOF through pores in the 2-4 nm range (which corresponds to a MW cut-off of about 20,000 for a hydrodynamically driven system) appears to be a challenging task since EOF decreases with decreasing pore size. The use of large pore membranes and charge trapping has the benefit of being easy to fill and use.

Clearly the sign of the electrophoretic mobility is important during the concentration of an analyte in front of a membrane. To determine if the magnitude of the electrophoretic mobility is important in effectiveness of the concentration, three peptides labeled with FITC were concentrated. The peptides, Gly-Ala, Gly-Ala-Val-Ser-Thr-Ala, and Pro-Leu-Gly-NH₂, have different electrophoretic mobilities which were measured to be about 3.9×10⁻⁴, 3.0×10⁻⁴ and 2.4×10⁻⁴ cm² V/Ns respectively. Although the magnitude of the electrophoretic mobilities are different, the sign of the electrophoretic mobilities are the same since they are all anionic at this pH. The concentration of these three FITC labeled peptides shows that the relative mobility greatly affects the speed of the concentration, but it does not affect the maximum concentration factor for analyte plugs of equivalent length. The peptides with smaller electrophoretic mobilities are not concentrated as rapidly, because they migrate slower down the channel. The overall speed for the concentration process is partially dependent on the design of the microfluidic device and the length of the channel. However, the efficiency of the concentration is not lower for slowing migrating compounds.

FIG. 9 shows a microfluidic device 5 of the present invention. The microfluidic device 5 was fabricated by sandwiching a polycarbonate ultrafiltration membrane 115 between a first PDMS piece 113 and a second 117 PDMS piece which have a three-dimensional microfluidic channel architecture. The vertical channels in the top and the bottom pieces are aligned so a solution can flow from the bottom to top channel after passing through the ultrafiltration membrane 115. The sample was introduced through an access port 112 and may be concentrated in front of the ultrafiltration membrane 115. The microfluidic device 5 further comprises a PDMS cover slide 111 and a PDMS bottom slide 119.

The microfluidic chip as shown in FIG. 9 may be fabricated as follows: the SU 8-25 photoresist (Microchemical, Newtown, Mass.) may be used to fabricate the 3-D master on a 10 cm diameter silicon wafer (Silicon Sense, Nashua, N.H.). Production of the 3-D photoresist master, required coating with 2 layers of photoresist. The first layer was about 40 μm thick and was made by spin coating at 1000 rpm for 30 s followed by baking at 90° C. on a hotplate for 20 min. After the coated wafer was cooled to room temperature, the first layer image was transferred onto the wafer by photolithography using UV lamp (365 nm) in a mask aligner for 30 s. The wafer was post-baked at 90° C. on a hotplate for 10 min, developed in SU 8-25 thinner for 30 s, and then in SU 8-25 developer until all unexposed photoresist was removed. A second, 100 μm thick layer of photoresist was formed by spin coating at 500 rpm for 10 s and baking for 2 hours at 90° C. After being cooled to room temperature, the wafer with the second layer of photoresist was exposed under a UV lamp with a second layer mask for 300 s. After the unexposed photoresist was removed, the wafer was heated at 150° C. for 30 min. For final preparation of the wafer, the surface was silanized with tridecafluoro-1,1,2,2-tetrahydroctyl-1-trichlorosilane under vacuum for 1 hour.

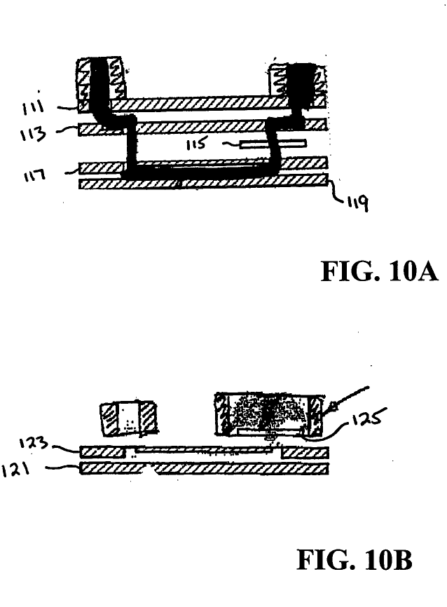

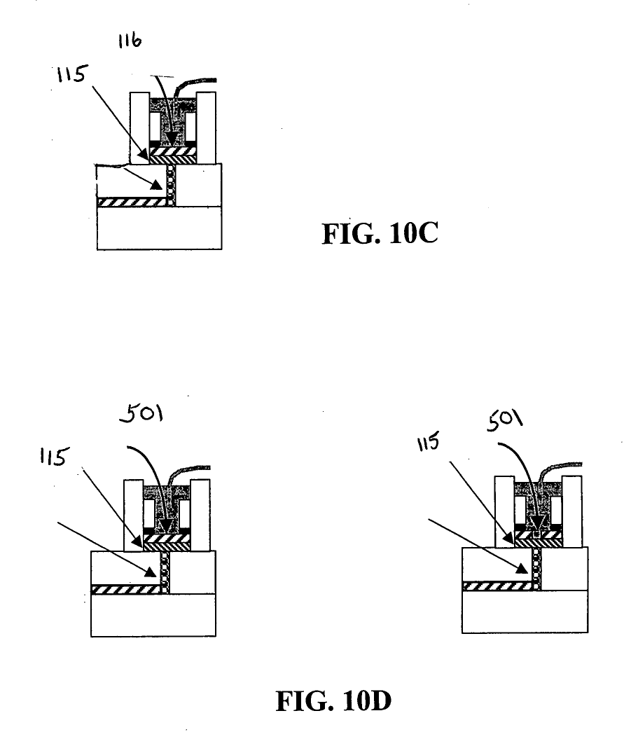

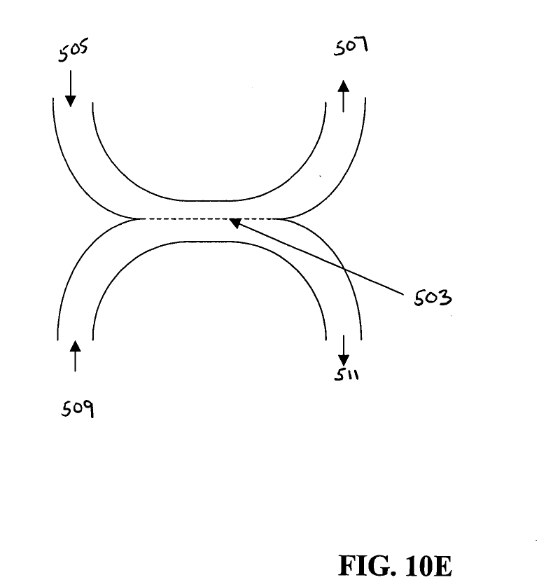

At 10:1 mixture of PDMS prepolymer and curing agent was mixed thoroughly and then degassed under vacuum until all bubbles are removed. To form the channels in the top and bottom substrates, the master wafer was spin coated with the uncured PDMS mixture 30 s at 1000 rpm, followed by curing at 90° C. hotplate for 15 min. The cured 100 μm thick PDMS layer was carefully removed from the master wafer. The top PDMS piece with the buffer reservoir access holes was cast on a mold machined from aluminum and nylon and cured at 70° C. for 2 hours. The bottom PDMS piece with no access holes or channels was cast on a silanized silicon wafer, and cured at 70° C. for 2 hours. The two pieces which formed the top half of the device and the two pieces that formed the bottom half of the device were bonded permanently. For permanent bonding, the PDMS parts were treated in a surface Plasma Cleaner (PDC-32 G, Harrick Scientific, Ossining, N.Y.), at 100 W for 1 min, and the exposed surfaces were pressed together within less than 1 min after their removal from the cleaner. The horizontal channels in the PDMS device were 100 μm and 40 μm in depth, while the vertical channels were 150 μm in diameter and 100 μm in height (measured from the surface of the substrate). The microfluidic device was completed by sandwiching a polycarbonate membrane (Harvard Bioscience, Holliston, Mass.) between the adhered top and bottom half of the PDMS chips as shown in FIG. 10.

A double T injector may be incorporated into the microfluidic device to enable injection of specific plug lengths. Variable injection volumes were accomplished by timing the injection into the channel. After injection of sample plugs having a finite volute, the flow was switched to the running buffer using standard methods.

In one aspect of the invention, the channels of the chip may be narrow and deep as compared to the conventional channels created through an acid-etch chip. In one aspect of the invention, the narrow and deep channels are incorporated into a glass chip by using a laser milling system. Such narrow and deep channels will provide less distortion of an applied electric field.

FIG. 10A and FIG. 10B show the path of a sample through the microfluidic device of FIG. 9. FIG. 10B shows a microfluidic device 5 comprised of glass. The microfluidic device 5 comprises a bottom slide 121, a bottom channel slide 123, and a membrane 125. With a glass microchip, the membrane 125 may be attached with an adhesive or the membrane 125 may be manually compressed against a surface with an appropriate mechanical force.

FIG. 10, C, FIG. 10D and FIG. 10E show various methods to attach and support a membrane on the microfluidic device of FIG. 9. In a preferred embodiment of the present invention, a nanocapillary array is incorporated into the microfluidic device 5. FIG. 1C shows the use of porous backing material 116 behind a nanocapillary array 115. The purpose of the backing material is to minimize the dead volume which can develop between the top of the microfluidic device 5 and the nanocapillary array. The nanocapillary array 115 often does not fit flatly and rigidly on top of the device creating a detrimental dead volume. A non-porous disk comprising a small hole could be used. However, it is difficult to align and immobilize the non-porous disk with the holes because they are about 50 microns in ID.

FIG. 10D illustrates fabrication of a supported nanocapillary array with non-porous backing material 501 by post-assembly drilling. First, non-porous backing material 501 is inserted. Second, a hole is drilled into the non-porous backing device 501 after the various pieces have been assembled. The method illustrated in FIG. 10D eliminates the difficult step of aligning a pre-drilled hole in the backing device with the vertical channel.

In addition to the methods of constructing a nanocapillary array illustrated in FIGS. 10C and 10D, the nanocapillary array 115 could be fabricated in-situ. As illustrated in FIG. 10E, deposition of a sol-gel membrane could be performed at the intersection 503 of 4 channels. These and other membranes can be formed between two discrete and immiscible phases (such as benzene and water). One channel 505 would be used to bring in a benzene reagent solution, and one adjacent channel 507 would be used to exit or remove the excess benzene solution. One of the other channels 509 would bring in the aqueous phase with its reagents and the last channel 511 (which also must be adjacent to the aqueous entrance channel) would form the aqueous exit channel. Deposition of a layer of aluminum could be achieved with a similar process to deposit Al metal from the reduction of A13+. The Al metal layer which would form in the middle of the intersection 503 could be converted to an aluminum oxide membrane in the channel.

FIG. 11 shows the concentration of 0.1 mM fluorescein in front of the 10 nm pore ultrafiltration membrane. The edges of the top and bottom channels have been drawn in for clarity, and the vertical channel is formed at the overlap of the two channels near the center of the images. (a) At 5 s the fluorescein solution has entered the field of view, but it is not visible because it is below the detection limit. (b) At 35 s the concentrated fluorescein is detectable under the ultrafiltration membrane. (c) At 100 s the concentration of the fluorescein has increased, and at 101 s the polarity was reversed. (d) At 120 s the concentrated plug is shown exiting the field of view.

FIG. 12 shows intensity profiles of the fluorescence intensity for the images shown in FIG. 11. The change in the fluorescence intensity during the fluorescein concentration is shown as a function of distance from the center of the ultrafiltration membrane. The plots were offset along the ordinate for better differentiation-the baseline or background intensities are equal for the raw data. (a) At 5 s the fluorescein plug has reached the field of view but is below the detection limit. (b), (c), and (d) were collected at 10 s, 26 s, and 53 s respectively and show the increasing concentration of fluorescein in front of the membrane. The polarity was reversed at 60 s. (e) At 67 s the initial reverse migration of the plug is showing a portion of the plug in both the horizontal and vertical channels. (f) and (g) At 80 s and 108 s shows the continued migration of the concentrated fluorescein plug out of the channel.

FIG. 13 shows the concentration of FITC labeled peptides which have different electrophoretic mobilities. It is shown that irregardless of the magnitude of the electrophoretic mobility the same final concentration or fluorescence intensity is reached, although the concentration is slower for the slower migrating analytes.

Downstream Separation Devices

In a currently preferred aspect, the microfluidic module 4 delivers peptides which are the products of proteolytic digestion of proteins traveling through the reaction channels 8 to a downstream separation module 14 prior to protein analysis. The downstream separation module 14 can comprise one or more of the separation columns described for the upstream separation device 2 above; however, preferably, the downstream separation module 14 comprises a capillary electrophoresis device comprising at least one separation path in communication with the microfluidic module 4 for providing a source of substantially separated digestion products.

Capillary electrophoresis is a technique that utilizes the electrophoretic nature of molecules and/or the electroosmotic flow of samples in small capillary tubes to separate sample components. Typically a fused silica capillary of 100 μm inner diameter or less is filled with a buffer solution containing an electrolyte. Each end of the capillary is placed in a separate fluidic reservoir containing a buffer electrolyte. A potential voltage is placed in one of the buffer reservoirs and a second potential voltage is placed in the other buffer reservoir. Positively and negatively charged species will migrate in opposite directions through the capillary under the influence of the electric field established by the two potential voltages applied to the buffer reservoirs. The electroosmotic flow and the electrophoretic mobility of each component of a fluid will determine the overall migration for each fluidic component. The fluid flow profile resulting from electroosmotic flow is flat due to the reduction in frictional drag along the walls of the separation channel. The observed mobility is the sum of the electroosmotic and electrophoretic mobilities, and the observed velocity is the sum of the electroosmotic and electrophoretic velocities.

In one aspect, a capillary electrophoresis system is micromachined on a device which is part of, or separate from, the protease digestion device 5 or interfacing device 5i described further below. Methods of micromachining capillary electrophoresis systems onto devices are well known in the art and are described in U.S. Pat. No. 6,274,089; U.S. Pat. No. 6,271,021; Effenhauser et al., 1993, Anal. Chem. 65: 2637-2642; Harrison et al., 1993, Science 261: 895-897; Jacobson et al., 1994, Anal. Chem. 66:1107-1113; and Jacobson et al., 1994, Anal. Chem. 66: 1114-1118.

To minimize sample loss, CE separations can be used which are capable of sample extraction. Fast CE separations in less then 1 second have been achieved, but these require extremely small injection volumes and short columns. To optimize the peak capacity and speed of a CE separation, it is necessary to determine the minimum column length for a given injection plug length (e.g., such as a sample plug). However, to maximize the peak capacity of an entire sample separation, an injection plug comprising one peak should not be mixed with peak(s)from a previous separation. If the optimized CE requires too long of a column and is too slow to avoid recombining peaks, then multiple CE separations can be run in parallel.

The dimensions of CE capillary match well with the channels of microfluidic devices in size. CE separations provide a more than adequate amount of sample for both MALDI-MS and ESI-MS/MS-based protein analyses (see, e.g., Feng et al., 2000, Journal of the American Society For Mass Spectrometry 11: 94-99; Koziel, New Orleans, La. 2000; Khandurina et al., 1999, Analytical Chemistry 71: 1815-1819. Therefore in one aspect, multiple parallel separation paths are provided which interface with multiple recipient channels 8r in a downstream microfluidic device 5i.

Preferably, electrophoretic concentration is used to counter the effects of band broadening and diffusion after polypeptide digestion.

Other downstream separation devices include, but are not limited to, micro high performance liquid chromatographic columns, for example, reverse-phase, ion-exchange, and affinity columns; however, these are less preferred.

It should be obvious to those of skill in the art that the exact configuration of the downstream separation module 14 can be varied. In one aspect, the downstream separation module comprises a separation medium and a capillary between the ends of which an electric field is applied. The transport of a separation medium in the capillary system and the injection of the sample to be tested (e.g., a sample band comprising peptides and/or partially digested polypeptides) into the separation medium can be carried out with the aid of pumps and valves but preferably by using electric fields which are suitably applied to various points of the capillary. Analysis time can be optimized by optimizing voltages, with higher voltages between the ends of a separating path generally resulting in an increase in speed. In a preferred aspect, voltages of about 10-1000 kV/cm are typically used resulting in separation times of about less than a few minutes.

The choice of buffers and reagents in the downstream separation module 14 are preferably optimized to be compatible with a downstream system with which it connects, such as the interfacing microfluidic module 4i and peptide analysis module 17, which are described further below. For example, as with the upstream separation module, ACN and solubilizing agents such as urea and guanidine can be used as buffer systems since these will not affect protein analyses such as MS. Similarly, as with the upstream separation module, CE can be combined with a solid-phase extraction (SPE) CE system.

Interfacing Microfluidic Module

In one aspect, the downstream separation module 14 is placed in communication with the peptide analysis module 17 by coupling the two devices using an interfacing microfluidic module 4i. This is particularly preferred when the downstream separation module 14 employs fast separation such as capillary electrophoresis (CE) as described above. While CE is well suited for the analysis of protein digests because of its high separation efficiencies, the narrow peak width representing separated peptides or partially digested polypeptides makes it difficult to perform subsequent tandem MS experiments needed to achieve high-quality MS/MS spectra.

Further, with fast separations such as CE, there is typically not enough time to obtain collision-induced dissociation (CID) on all of the ions eluting from a column, because the flow rate of injection into the peptide analysis module 17 is dictated by the flow rate of the separation. For example, currently with a standard capillary LC-MS run, only about 10% of the total LC-MS run time during active peptide elution is spent on detecting and trapping peptides by MS while most of the time is spent on loading and re-equilibrating the LC column. For CE separations of peptides, the amount of time spent on sample loading and column rinsing is decreased greatly but is still substantial.

Flow modulation techniques have been developed for CE (see, e.g., Figeys et al., 1999, Anal. Chem. 71: 2279-228) and LC (see, e.g., Davis et al., 1995, Anal. Chem. 67: 4549-4556; Davis et al., 1997, J American Society for Mass Spectrometry 8: 1059-1069), but degrade the quality of the separation and can modulate the flow only over a small range. The small range over which the flow can be modulated is due to: i) the degradation of the ongoing separation and ii) the need to use an electrospray capillary and tip for delivery into an MS device with an inside diameter large enough to accommodate both normal and reduced flow rates.

To circumvent these difficulties, in a preferred aspect, an interfacing microfluidic module 4i (shown in FIG. 1A) is used to inject sample bands into a peptide analysis module 17 such as an MS/MS device. The sample bands represent fractions comprising substantially purified peptides and/or partially digested polypeptides obtained after digestion of proteins in the microfluidic module 4 and the ensuing separation of these products using the downstream separation module 14. The interfacing microfluidic module 4i can have a similar structure as the on-device digestion module 4 without the first solid phase.

The interfacing microfluidic module 4i according to the invention decouples the separation process occurring in the downstream separation module 17 from the protein analysis process in time to achieve lower limits of detection by performing one or more of the following functions: (1) storing the substantially purified peptides or partially digested polypeptides in reaction channels 8 until analysis; (2) electrophoretically concentrating the peptides/partially digested polypeptides prior to analysis; and (3) injecting the peptides/partially digested polypeptides into the peptide analysis module 17 (e.g., an MS system) with a delivery element 22 such as an electrospray source while retaining or eliminating eluent not containing peptides/partially digested polypeptides. Decoupling separation from detection and analysis provides more time to obtain CID spectra on all of the ions eluted from the downstream separation module 17 without causing an increase in overall analysis time.

The interfacing microfluidic module 4i can be fabricated using methods similar to those used to create the on-device digestion microfluidic module 4. Preferably, one or more electrodes are shielded from the overlying substrate to electrically isolate fluid flow within the device. However, in aspects where the overlying substrate 6i is not glass, any or all of the electrodes may be alternatively, or additionally, formed on the surface of the overlying substrate proximate to the device 5i.

The interfacing microfluidic device 5i can comprise more than one channel 8i and in one aspect, a channel geometry similar to that shown in FIG. 1A for the protease digestion device 5 is employed. Constraints on channel geometry are similar to those described above for the protease digestion device 5. However, the channel number and geometry of the interfacing device 5i also is influenced by the operating parameters of the downstream peptide analysis module 17 with which it is coupled. For example, a large number of channels 8i (e.g., about 32-64) is useful to evaluate post-translation modifications which are present in low stoichiomteric ratios in a sample where unmodified peptides are at high concentration and modified peptide(s) are present at low concentrations since multiple channels 8i can facilitate parallel analysis by the peptide analysis module 17.

Directing sample bands from the downstream separation module 14 to different channels 8i of the device 5i is a simple task if they can be held for processing until the end of a subsequent separation (i.e., elution of a next sample band into channel(s) of the device 5i). However, if sample analysis must begin before the end of a subsequent separation then the task is more complex. The method proposed herein relies on a physical separation between some of the sample bands or peaks representing digested, purified peptides which have been separated by the downstream separation module 14. There must be some gap between bands or peaks to begin moving the collected bands into the different channels of the device 5i. In one aspect, a spatial separation between bands or peaks is attained by moving the bands/peaks past an electrode that can isolate them. At this point, the bands/peaks can be manipulated independently of eluent from the downstream separation module (e.g., by directing eluent not comprising peptides or partially digested polypeptides to reservoirs within the device). However, if the separation is so full of peaks that there are no gaps, then there is enough sample that all of the peaks do not need to be analyzed.

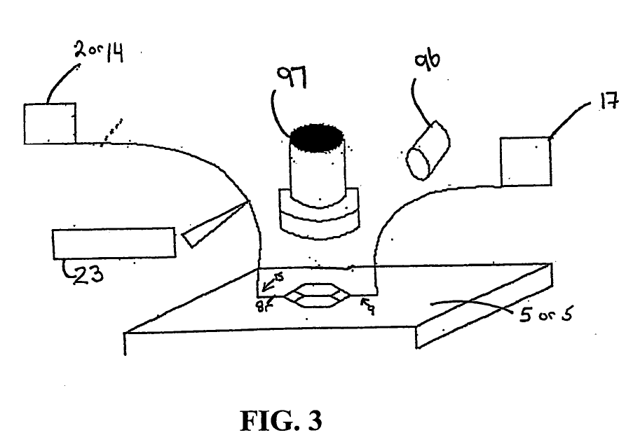

The velocity of sample elution from the downstream separation module 14 can be calculated and used to predict the velocity of fluid flow through channel(s) 8i of the interfacing device 5i. Accurate assessment of velocity is required for properly timed control of current through electrodes in communication with the device 5i in order to control flow of sample through the device 5i. As shown in FIG. 3, an arc-lamp 96 and CCD camera 97 is used to monitor the accuracy and reproducibility of sample band transport to the various channels of the device. Similar fluorescence detectors have been designed to image separations in wide channels (Liu et al., 1996, Anal. Chem. 68: 3928-3933; Hietpas et al., 1981, Anal. Chem. 69: 2292-2298). After optimal sample flow is determined, control of current through the various electrodes of the device may be implemented without the use of a CCD camera, e.g., by pre-programming proper current/voltage parameters and temporal sequences into the processor 18 of the system 1. A similar arrangement can be used to monitor and optimize flow in the protease digestion device 5.

In another aspect, the optical coupling of detectors 23 to the on device is used to determine when a sample has arrived in a channel. In one aspect, a voltage control system in electrical communication with electrodes of the interfacing microdevice 5i uses the input from an optical detector 23 at the device 5i entrance to determine where sample peaks are, and uses this data as the basis for flow control. For example, in one aspect, a system processor 18 in communication with the voltage control system implements a voltage control program to perform real-time peak recognition to determine the beginning and end of each sample band and the position of a sample band on the device 5i.

In addition to transporting sample bands, the interfacing device 5i can be used to hold and/or concentrate and/or focus peptides and/or partially digested polypeptides before they are injected into the peptide analysis module 17. This is desired particularly where sample channels 8 in the protease digestion device 5 are in a substantially parallel configuration (e.g., as in FIG. 2A) since a second solid phase generally cannot be used to concentrate samples in this embodiment.

In one aspect, sample concentrating is performed at an interface between two different conductivity buffers within one or more channels 8i of the device 5i to achieve a concentration factor of about ten or more. Other methods such as transient iso-electric focusing (IEF) can be used, preferably without the use of carrier ampholytes which tend to increase background and increase detection limits (i.e., lower detection sensitivity) (see, e.g., as described by Koziel et al., New Orleans, La. 2000). For example, a temperature gradient can be formed by passing a current through a solution in a channel 8i having a temperature gradient in cross-sectional area. The temperature gradient forms a pH gradient enabling efficient isoelectric focusing. Microfluidic systems are extremely well suited for such electrophoretic concentration methods as buffer exchange can be performed on the device.

While overloading sample can disrupt the pH gradient where IEF has been used in the downstream separation module 14, this is not a large concern in the interfacing module 4i because only one band is being focused in the interfacing device 5i. By focusing in the interfacing module 4i, a second dimension separation can be provided to further resolve sample bands which were not separated by a first dimension in the downstream separation module 14. Since bands from the first dimension are not recombined in the interfacing module 4i, this provides a true two-dimensional separation and can resolve peaks co-eluting from the downstream separation module 14.

Microdialysis membranes (Liu et al., 1998, Analytical Chemistry 70: 1797-1801; Xiang et al., 1999, Analytical Chemistry 71: 1485-1490; Xu et al., 1998, Analytical Chemistry 70: 3553-3556) and sieving frits (Khandurina et al., 1999, Analytical Chemistry 71: 1815-1819) also can be incorporated onto a device 5i, making it possible to perform buffer exchange without sample dilution. Effective on-device concentration could be extremely beneficial to improving detection limits, as the signal-to-noise ratio is directly proportional to the concentration of sample. Not only does on-device concentration give a greater increase in signal-to-noise than mathematical operations such as ensemble averaging, but it also shortens the time needed for analysis by compressing the sample band length. As discussed above, the interfacing microfluidic module 4i does not introduce any dead volume or sample dilution from eluent that would negate the effects of any attempted concentration.

In one aspect, on-device concentration immediately prior to protein analysis is used to minimize the effects of diffusion and is achieved by varying pH in one or more channels 8i of the device 5i. For example, holding the sample in a channel 8i for 20 minutes will broaden a sample plug by just 1 mm (assuming a diffusion coefficient of 1×10⁻⁶ cm²/s). A static discontinuous buffer front can be formed by ionic transport through a dialysis membrane or frit sandwiched between the device 5i and its overlying substrate 6i. With a decrease in pH in the channel, the electrophoretic velocity of peptide analytes in the channel 8i decreases, giving rise to a concentrating or stacking effect. By running a buffer with low pH through one or more of the channels 8i, the pH of a sample stream can be lowered directly before it is delivered into the peptide analysis module 17 (e.g., sprayed into an MS device) (see, e.g., as described in Liu et al., 1988, Analytical Chemistry 70: 1797-1801; Xiang et al., 1999, Analytical Chemistry 71: 1485-1490; Xu et al., 1998, Analytical Chemistry 70: 3553-3556; Yang et al., 1998, Analytical Chemistry 70: 4945-4950; and Jacobson et al., 1994, Anal. Chem. 66: 1107-1113; Timperman et al., 1995, Anal. Chem. 67: 139-44, for example).

In addition to improving the limits of detection of the peptide analysis module 17 by electrophoretic concentration of samples, buffer exchange can be used to provide an optimum pH for sample delivery from the interfacing module 4i to the peptide analysis module 17.

Preferably, the interfacing module 4i provides a mechanism to switch from a pH which is optimal for an upstream component of the system 1, such as the downstream separation module 14, to a pH which is optimal for the particular peptide analysis module 17 used. For example, with CE, the optimum pH for separation is near a neutral pH (Nice, 1996, Biopolymers (Peptide Science) 40: 319-341) while the best sensitivity for ESI-MS is obtained with a pH between 2 and 3. Therefore, in one aspect, as shown in FIG. 6, appropriate high and low pH conditions are switched on and off to change the interfacing module 4i from a sample loading mode to a holding and focusing mode, and from a holding and focusing mode to a transport mode which directs sample towards the peptide analysis module 17.

In one aspect, a side channel can be provided (not shown) which provides a pH altering solution comprising ions for regulating pH. Preferably, the side channel is electrically isolated from other channels 8 of the device and the pH altering solution is introduced selectively into one or more other channels of the device by selectively applying a voltage at the side channel and the one or more other channels of the device at a desired time period.

In one aspect, the interface microfluidic device 5 is coupled to the peptide analysis module 17. In a most preferred version, an electrospray system formed by a capillary coupled to an exit channel in the microdevice 5i (not shown) which is in proximity to a sampling orifice of the peptide analysis module 17. An electrospray is produced when a sufficient electrical potential difference is applied between a conductive or partly conductive fluid exiting the capillary orifice (e.g., such as a fluid containing substantially purified peptides received from the downstream separation module 14) and an electrode so as to generate a concentration of electric field lines emanating from the tip or end of a capillary. When a positive voltage is applied at the sampling orifice of a peptide analysis module 17 (e.g., such as the ion-sampling orifice of a mass spectrometer), the electric field causes positively-charged ions in the fluid to migrate to the surface of the fluid at the tip of the capillary. Similarly, when a negative voltage is applied, the electric field causes negatively-charged ions in the fluid to migrate to the surface of the fluid at the tip of the capillary.

When the repulsion force of the solvated ions exceeds the surface tension of the fluid sample being electrosprayed, a volume of the fluid sample is pulled into the shape of a cone, known as a Taylor cone which extends from the tip of the capillary (see, e.g., Dole et al., 1968, Chem. Phys. 49: 2240 and Yamashita and Fenn, 1984, J Phys. Chem. 88: 4451). The potential voltage required to initiate an electrospray is dependent on the surface tension of the solution (see, e.g., Smith, 1986, IEEE Trans. Ind Appl. IA-22: 527-535). The physical size of the capillary determines the density of electric field lines necessary to induce electrospray. The process of electrospray ionization at flow rates on the order of nanoliters per minute has been referred to as “nanoelectrospray”. However, the term “electrospray” shall be used to encompass nanospray herein.

Electrospray into the ion-sampling orifice of peptide analysis module can produce a quantifiable response in a detector component of the peptide analysis module due to the presence of analyte molecules (e.g., substantially purified peptides) present in the liquid flowing from the capillary. Electrospray devices are known and described in the art (see, e.g., Wilm and Mann, 1996, Anal. Chem. 68: 1-8; Ramsey et al., 1997, Anal. Chem. 69: 1174-1178; Xue et al., 1997, Anal. Chem. 69: 426-430).

Nozzles also can be used to form electrospray systems. For example, Desai et al., Jun. 16-19, 1997, International Conference on Solid-State Sensors and Actuators, Chicago, 927-930, describes the generation of a nozzle on the edge of a silicon microdevice and applying a voltage to the entire microdevice. In one aspect, a nozzle is used which has an inner and an outer diameter and is defined by an annular portion recessed from an ejection surface. The annular recess extends radially from the outer diameter. The tip of the nozzle is co-planar or level with and does not extend beyond the ejection surface and thus the nozzle is protected against accidental breakage. The nozzle can be etched by reactive-ion etching and other standard semiconductor processing techniques (see, e.g., as described in U.S. Pat. No. 6,245,227).

However, preferably, the electrospray system comprises a capillary. In one aspect, the capillary is coupled to the overlying substrate 6i of the interfacing microfluidic module 4i through an opening in the overlying substrate 6i which connects to an exit channel in the interfacing device 5i. Preferably, the capillary is at an angle with respect to the surface of the interfacing microfluidic device 5i (e.g., such as a 45° C. to 90° C. angle). The electrospray system is placed about 0-10 mm, and preferably, about 0-2 mm from the sampling orifice of the peptide analysis module 17.

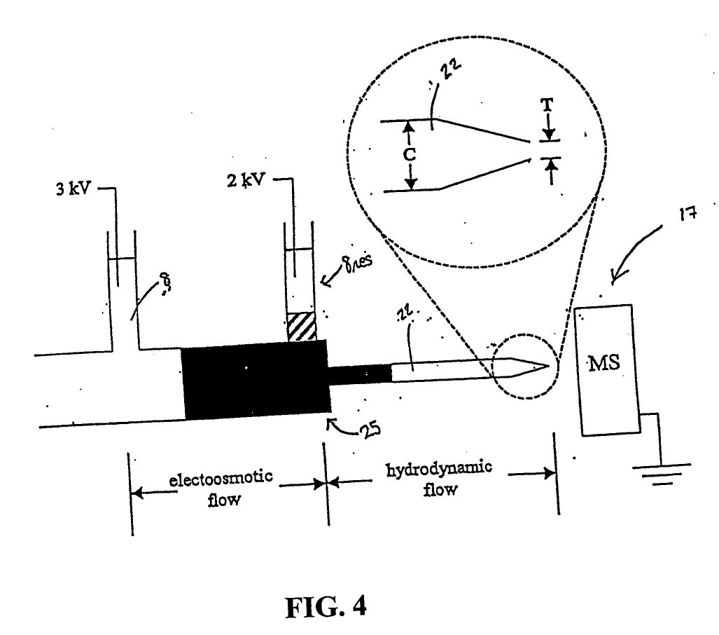

FIG. 4 shows a schematic diagram showing the connection between a transport channel on an interfacing microfluidic device 5i (large ID) and a delivery element 22 which is an electrospray spray capillary (small ID). The black shape represents a sample band as it is transferred to the capillary. The voltage between the two electrodes shown in communication with the device 5i creates an electroosmotic flow which forces sample solution through the nanospray capillary 22. The voltage drop across the capillary 22 is negligible; so there is no electrophoretic flow in this region. A frit or flow restricting or balancing material or channel configuration that retards flow (cross-hatched box) retards the flow of solution to help force solution through the electrospray capillary 22. The detailed inset clarifies the difference between the electrospray capillary 22 internal diameter (ID) “C” and the electrospray tip (ID) “T”. In a currently preferred aspect, the electrospray capillary ID is about 10 μm.

However, in a currently preferred embodiment, to avoid reverse focusing or a dilution effect, the sample band is pushed onto the spray capillary 22 by electroosmotic pumping. An electroosmotic flow pump (EOF pump) utilizes electroosmotic pumping of fluid in one channel or region to generate a pressure-based flow of material in a connected channel (see, e.g., as described in U.S. Pat. No. 6,171,067). For electroosmotic pumping, there is no voltage drop across the spray capillary 22, and the EOF to force the solution onto the spray capillary can be generated in the channel 8i immediately preceding the capillary 22. The design for a nanospray interface (e.g., as shown in FIG. 4) can be optimized by adjusting the length and volume of sample in an EOF pump region and in regions free of electric fields in the device 5i. Low flow rates can be obtained using EOF pumps and flow rates can be controlled by controlling the applied voltage at different regions/channels 8i of the device 5i. Additional non-EOF pumping systems are described by Feng et al., 2000, Journal of the American Society For Mass Spectrometry 11: 94-99. For example, hydrodynamic flow systems can be used, as discussed above.

It is important to move sample into the electrospray tip without providing an excessive amount of band broadening. However, a greater amount of band broadening can be tolerated in the system 1 according to the invention than in an analytical separation because sample plug length will be very long with respect to the inside diameter of the spray capillary 22. Sample bands can be transferred from a channel 8i of the device 5i to the capillary 22 electrophoretically, for example, by applying a spray voltage directly at the tip of the capillary 22 to create a potential drop across both the channel 8i and the capillary 22.

Electroosmotic pumping is preferred for rapid delivery of a peptide mixture into a peptide analysis module 17 directly from the interfacing device 5, especially where the peptide analysis module obtains and analyzes data quickly. For example, Fast ESI-TOF machines can collect spectra at rates of 4 Hz (Liu et al., 1998, supra). Peptide mass fingerprinting is more complicated with ESI instruments, but also has been demonstrated to work (see, e.g., Xiang et al., 1999, Analytical Chemistry 71: 1485-1490; Xu, et al., 1998, Analytical Chemistry 70: 3553-3556). This seamless approach would eliminate the spotting and dry-down which is needed for peptide analysis modules such as MALDI and avoids the competitive ionization problems encountered with MALDI that limit the observable number of peptides.

Interfacing with a MALDI device is still straightforward, as automated spotters that connect capillaries and MALDI targets have been developed (see., e.g., Figeys et al., 1998, Electrophoresis 19: 2338-2347). In a particularly, preferred aspect, for example, where post-translational modifications are being evaluated, a small amount of protein solution can be rapidly forced through the various modules of the system 1 such that a protein passes undigested through the protease digestion module 4 and the precise protein molecular mass can be recorded along with a precise peptide mass map when peptide samples are subsequently delivered to the peptide analysis module 17.

In some instances, protein analysis time can be extended and detection limits improved by decreasing the flow rate into a peptide analysis module 17 such as an MS device. As discussed above, electrospray is concentration sensitive (Kebarle et al., 1997, supra) and usually the flow rate into the MS is dictated by an upstream separation system, and is therefore not optimized for MS detection. For example, typically, capillary HPLC-MS is operated at flow rates of about 200 nL/min (see, e.g., Gatlin et al., 1998, Analytical Biochemistry 263: 93-101) and CE-MS is operated at flow rates or about 25 nL/min. To obtain a 20-fold reduction in flow rate, the electrospray must be able to operate at flow rates of 10 nL/min for capillary HPLC-MS and at about 1 nL/min for CE-MS. Such flow rates are low, but stable electrospray has been obtained for flow rates down to 0.5 nL/min (see, e.g., Valaskovic et al., 1995, Analytical Chemistry 67: 3802-3805). Because the interfacing module 4i extends the analysis period of the peptide analysis module 17 into the “dead-time” between the end of one separation and the beginning of the next (e.g., during the time between re-equilibration of the downstream separation module 14 and sample injection), an electrospray source can be used with a lower volumetric flow. Since electrospray is concentration dependent (see, e.g., Banks et al., 1996, supra; Karger, 1996, supra; Kebarle et al., 1997, supra), no loss in signal will be observed.

Obtaining very low flow rates (˜0.5 nL/min) at a nanospray source is more dependent on the inside diameter of the capillary 22 than on the inside diameter of the spray tip (Valaskovic, 1995, supra). Therefore, in a preferred aspect, a capillary 22 with a small inside diameter (5-10 μm) is used to interface the interfacing microdevice 5i with the MS system (see, FIG. 6). Preferably, the diameter of the capillary 22 is at least smaller than the diameter of the channel 8i of the interfacing device 5i which delivers sample to the capillary 22. In one aspect, the capillary 22 is interfaced directly with an about 50 μm channel 8i on the device 5i.

In a further aspect, the interfacing microfluidic module is physically separated from a plurality of nanospray needles which can be aligned for transfer of solution subject to an operator's control (directly or through a processor), using a rotary system similar to one developed for loading microfabricated capillary arrays (see, e.g., Scherer et al., 1999, Electrophoresis 20: 1508-1517). Recently, arrays of electrospray needles have been fabricated on silicon devices (see, e.g., Zubritsky et al., 2000, Anal. Chem. 72: 22A; Licklider et al., Anal. Chem. 72: 367-375).

Each sample band stored in a channel and delivered into the peptide analysis module 17 is not necessarily pure. However, unresolved peaks are common in systems such as capillary LC-MS/MS and all must be analyzed in a very short time. One great advantage of the system 1 according to the invention is that the nanospray interface allows adequate time to analyze unresolved peptides. Separation and/or focusing by the downstream separation module 14 and/or interfacing module 4i is a crucial step because sample concentration can be increased by orders of magnitude through sample extraction and concentration. The extraction and concentration capabilities of the system 1 allow a peptide analysis module 17 such as an MS device to analyze a peptide solution of much higher concentration.

Peptide Analysis Modules

The peptide analysis module refers to a device which provides chemical or physical analysis of the sample, and could be more generally called the structural analysis module. Specifically peptides are the most preferred analyte and therefore the peptide analysis module has been the most preferred structural analysis module. However, the microfluidic system could be applied to more analytes than polypeptides and therefore the peptide analysis module is more generally a structural analysis module. The peptide analysis module 17 is preferably some form of mass spectrometer (MS) device comprising an ionizer, an ion analyzer and a detector. Any ionizer that is capable of producing ionized peptides in the gas phase can be used, such as anionspray mass spectrometer (Bruins et al., 1987, Anal Chem. 59: 2642-2647), an electrospray mass spectrometer (Fenn et al., 1989, Science 246: 64-71), and laser desorption device (including matrix-assisted desorption ionization and surfaced enhanced desorption ionization devices). Any appropriate ion analyzer can be used as well, including, but not limited to, quadropole mass filters, ion-traps, magnetic sectors, time-of-flight, and Fourier Transform Ion Cyclotron Resonance (FTICR). In a preferred aspect, a tandem MS instrument such as a triple quadropole, ion-trap, quadropole-time-of flight, ion-trap-time of flight, or an FTICR is used to provide ion spectra.

In one aspect, molecular ions (e.g., daughter ions) generated by ionization of peptides from the delivery element of the interfacing module 22 (e.g., such as an electrospray) are accelerated through an ion analyzer of the peptide analysis module 17 as uncharged molecules and fragments are removed. Preferably, the ion analyzer comprises one or more voltage sources (e.g., such as electrodes or electrode gratings) for modulating the movement of ions to a detector component of the peptide analysis module. Daughter ions will travel to the detector based on their mass to charge ratio (m/z) (though generally the charge of the ions will be the same). In a preferred aspect, the detector produces an electric signal when struck by an ion.

Timing mechanisms which integrate those signals with the scanning voltages of the ion analyzer allow the peptide analysis module 17 to report to the processor 18 when an ion strikes the detector. The processor sorts ions according to their m/z and the detector records the frequency of each event with a particular m/z. Calibration of the peptide analysis module 17 can performed by introducing a standard into the module and adjusting system components until the standard's molecular ion and fragment ions are reported accurately. Preferably, the peptide analysis module 17 in conjunction with the processor 18, plots a product ion spectra which corresponds to a plot of relative abundance of ions produced vs. mass to charge ratio. The detected product ions are formed by isolating and fragmenting a parent ion (that is typically the molecular mass of a peptide molecule) in the peptide analysis module (e.g., a mass spectrometer).

Generally, peptides typically fragment at the amide bond between amino acid residues and peaks correspond to particular amino acids or combinations of amino acids. While there may be additional peaks (ions) present in the product ion spectra, many of these other peaks can be predicted and their presence explained by comparison with spectral data of known compounds (e.g., standards). Many different processes can be used to fragment the parent ion to form product ions, including, but not limited to, collision-induced dissociation (CID), electron capture dissociation, and post-source decay.

Analysis of product ion spectra will vary depending upon the particular type of peptide analysis module 17 used.

For high throughput identification of polypeptides, matrix assisted laser-desorption ionization mass spectrometry (MS) peptide finger printing is the method of choice. Although this method is fast, it requires protein database matching and provides the least detailed information. When more detail is needed, ionization tandem mass spectrometry (ESI-MS/MS) is the method of choice (see, e.g., Karger et al., 1993, Anal Chem. 65: 900-906). MS/MS is capable of giving amino acid level sequence information and is required for de novo sequencing and analysis of post-translational modifications. The development of automated database searching programs to directly correlate MS/MS spectra with sequences in protein and nucleic acid databases has greatly increased throughput. New hybrid instruments are being developed to combine MALDI with MS/MS are being developed to combine MALDI with MS/MS to combine speed of analysis with amino acid sequence information. It should be apparent to those of skill in the art that as MS tools evolve new interfaces can be developed to couple microfluidic devices according to the invention with either MALDI or HIS sources.

In one aspect, the spectra obtained by the peptide analysis module 17 are searched directly against a protein database for identification of the polypeptide from which the peptide originated. However, preferably, the peptide analysis module 17 obtains sequence information directly from spectra obtained by the peptide analysis module 17 without the use of a protein or genomic database. This is especially desirable when the protein to be identified is not in a protein database. Therefore, in one aspect, rather than performing a search function to compare peptide sequences to a protein database, the processor 18 implements an algorithm for automated data analysis of spectra obtained from the peptide analysis module 17.

Preferably, the peptide analysis module 17 facilitates this interaction by isolating daughter ions (MS² ions) obtained from parent ions sprayed into the module (e.g., via an electrospray) and further isolating and fragmenting these to obtain granddaughter ions (MS³ ions) to thereby obtain MS³ spectra. For these types of analyses, ion-trapping instruments such as Fourier transform ion cyclotron resonance mass spectrometers and ion trap mass spectrometers are preferred.

MS³ spectra generally comprise two classes of ions: ions with the same terminus as daughter ions (MS² ions) and ions derived from internal fragments of peptides (some of this latter class include C-terminal residues). By identifying peaks that are common to both MS² and MS³ spectra (e.g., contained with an intersection spectrum), a partial sequence of the peptide can be read directly from the intersection spectrum based on the differences in mass of the major remaining ions. Obtaining MS³ spectra of many daughter ions of a peptide will generate many intersection spectra which in turn will generate many partial sequences of different areas of a peptide. Partial sequences can be combined to obtain the complete sequence of the peptide by correlating experimentally acquired spectra with theoretical spectra which are predicted for all of the sequences in a database. A fast Fourier transform can be used to determine the quality of the match. In a preferred aspect, detection limits are improved further by ensemble averaging of many spectra (Wilm, 1996, Analytical Chemistry 68: 1-8).

The speed of protein analysis will depend mainly on the voltage used to mobilize the samples, geometry of the channels in the interfacing microfluidic device 5i, and the number of scans used by the protein analysis system for acquisition of data relating to a sample band. The number of scans can be optimized using methods routine in the art. For example, for ensemble averaging, the increase in signal-to-noise ratio is equal to the square root of the number of scans averaged, so at larger numbers of scans, there will be diminishing returns. Since increasing the number of scans will also increase analysis time, there will be an optimum number of scans to average. This number will be determined by the efficiency at which the system can load the samples into the electrospray/nanospray capillary and the complexity of the sample.

Higher concentration samples will contain more detectable peaks and will require less averaging. Because lower concentration samples will contain fewer peaks, there will be more time to acquire scans. An optical detection system, such as the one described above, can be used to measure the complexity of a sample before it reaches the MS and this information can be used to determine the optimum scan number.

The peptide analysis module 17 preferably compares the results of multiple runs of sample through the system 1. Thus, in one aspect, the results of one run are compared to the results of another run utilizing the same protein or peptide sample. In another preferred aspect, the protein analysis compare multiple runs of sample which have been exposed for various periods of time to proteases within the protease digestion module 4 enabling analysis of undigested, partially digested, and completely digested proteins or polypeptides in the sample.

In a preferred aspect, the peptide analysis module 17 identifies post-translational modifications in cellular proteins. Generally, post-translational modifications may be classified into four groups, depending upon the site of chemical modification of the protein. For example protein modifications may involve the carboxylic acid group of the carboxy terminal amino acid residue, the amino group of the amino terminal amino acid residue, the side chain of individual amino acid residues in the polypeptide chain, and/or the peptide bonds in the polypeptide chain. The modifications may be further sub-grouped according to distinct types of chemical modifications, such as phosphorylation, glycosylation, acylation, amidation and carboxylation. Using MS, peptide ions are fragmented into peptide fragment ions which are selected and further fragmented to yield information relating to the nature and site of a modification.

Other methods could be used for the structural analysis model. An example of another system which provides chemical or physical information concerning the analyte is nuclear magnetic resonance (NMR).

Detectors

In one aspect, as shown in FIG. 1A, detectors 23 are placed at various flow points of the system 1 to enable a user to monitor separation efficiency. For example, one or more spectroscopic detectors 23 can be positioned in communication with various channels, outputs and/or modules of the system 1. Spectroscopic detectors rely on a change in refractive index, ultraviolet and/or visible light absorption, or fluorescence after excitation of a sample (e.g., a solution comprising proteins) with light of a suitable wavelength.

In a preferred aspect, sample bands comprising substantially separated proteins (e.g., obtained after passage through the upstream separation module 2) or substantially purified peptides (e.g., obtained after passage through the microfluidic module 4 and the downstream separation module 14) are actively sensed by optical detectors which recognize changes in a source light (e.g., such as a ultraviolet source) reacting with the sample bands. In response to such changes the detectors produce one or more electrical signals which are received and processed by processors 18 in electrical communication with the detectors.

In one aspect, a detector 23 is provided which detects the native fluorescence of polypeptides and peptides which pass through various modules of the system 1. Such fluorescence arises from the presence of tryptophan, tyrosine, and phenylalanine residues in these molecules. Preferably, the detector 23 comprises a laser (e.g., a 210-290 nm laser) for excitation of a sample band as it passes within range of detection optics within the system and collects spectra emitted from the polypeptides, partially digested polypeptides, or peptides within the sample band in response to this excitation. The detector 23 can comprise lens or objectives to further focus light transmitted from the laser or received from polypeptides/peptides.

Preferably, the detector 23 transmits signals corresponding to the emission spectra detected to the processor 18 of the system 1 and the processor records the time and place (e.g., module within the system) from which the signals are obtained. Detectors for detecting native fluorescence of polypeptides and peptides and which are able to spectrally differentiate at least tryptophan and tyrosine are known in the art, and described, for example in Timperman et al., 1995, Analytical Chemistry 67(19): 3421-3426, the entirety of which is incorporated by reference herein. As discussed above, the detector 23 can be used to monitor and control sample flow through the system 1.

In a particularly preferred aspect, a detector is integrated into module within the system. For example, a UV or thermal lens detector can be used and integrated into either or both the protein digestion module 4 or the interfacing module 4i. Recent advancements have been made with both detection systems, and limits of detection for these systems are in the low nanomolar range (see, e.g., Culbertson et al., 1999, Journal of Microcolumn Separations 11: 652-662. In one aspect, a UV detection system with a multi-reflection cell is integrated into a device within the system (see, e.g., as described in Salimi-Moosavi et al., 2000, Electrophoresis 21: 1291-1299). Extremely low yoctomole detection limits have been achieved on-device with a thermal-lens detector (see, e.g., Sato et al., 1999, Analytical Sciences 15: 525-529).

In a preferred aspect of the invention, as shown in FIG. 1A, a detector 23 is placed in optical communication with the separation channel between the upstream separation module 2 and the recipient channel of the microfluidic device 5. The detector detects sample bands delivered by the upstream separation module to the device 5 and the processor 18 in response to the signals received from the detector 23 performs a background subtraction which eliminating background electrolyte signal as sample bands are directed to one of the reaction channels 8 in the device 5. “Cutting” the sample bands allows the peptide analysis module 17 to spend more of its time on sample analysis and less on analysis of background electrolytes. For low concentration protein samples, a very small fraction of the time (<2%) actually is spent analyzing the sample.

Preferably, the protein analysis system 17 includes its own detector (not shown) which detects spectral information obtained from peptides being analyzed by the system 17. For example, the protein analysis detector can detect various charged forms of peptide ions as they pass through a peptide analysis module 1, such as an ESI MS/MS system.

As discussed above, in one aspect, one or more detectors 23 (including the protein analysis detector) are electrically linked to a processor 18. As used herein, the term “linked” includes either a direct link (e.g., a permanent or intermittent connection via a conducting cable, an infra-red communicating device, or the like) or an indirect link such that data are transferred via an intermediate storage device (e.g. a server or a floppy disk). It will readily be appreciated that the output of the detector should be in a format that can be accepted by the processor 18.

It should be obvious to those of skill in the art that a variety of detectors 23 can be selected according to the types of samples being analyzed. Detectors 23 additionally can be coupled to cameras, appropriate filter systems, and photomultiplier tubes. The detectors 23 need not be limited to optical detectors, but can include any detector used for detection in liquid chromatography and capillary electrophoresis, including electrochemical, refractive index, conductivity, FT-IR, and light scattering detectors, and the like.

Processors

In a preferred aspect, a system processor 18 is used to control flow of proteins/peptides through the system 1, e.g., based on data obtained from detectors placed at various positions in the system. In a preferred aspect, the interfacing module 4i of the system I uses this control to increase the amount of time the peptide analysis module 17 actually spends analyzing sample and obtaining sequence information.

As used herein, “a system processor” refers to a device comprising a memory, a central processing unit capable of running multiple programs simultaneously, and preferably, a network connection terminal capable of sending and receiving electrical signals from at least one non-system device to the terminal.

The system processor 18 is in communication with one or more system components (e.g., modules (2, 14, 4, 4i 17), detectors 23, computer workstations and the like) which in turn may have their own processors or microprocessors. These latter types of processors/microprocessors generally comprise memory and stored programs which are dedicated to a particular function (e.g., detection of fluorescent signals in the case of a detector 23 processor, or obtaining ionization spectra in the case of a peptide analysis module 17 processor, or controlling voltage and current settings of selected channels on a device in the case of a power supply connected to one or more devices) and are generally not directly connectable to the network.

In a preferred aspect, the system processor 18 is in communication with at least one user device comprising a display for displaying a user interface which can be used by a user to interface with the system 1 (i.e., view data, set or modify system 1 parameters, and/or input data). The at least one user device can be connected to an inputting device such as a keyboard and one or more navigating tools including, but are not limited to, a mouse, light pen, track ball, joystick(s) or other pointing device.

The system processor 18 integrates the function of processors/microprocessors associated with various system components and is able to perform one or more functions: of data interpretation (e.g., interpreting signals from other processors/microprocessors), data production (e.g., performing one or more statistical operations on signals obtained), data storage (e.g., such as creation of a relational database), data analysis (e.g., such as search and data retrieval, and relationship determination), data transmission (e.g., transmission to processors outside the system such as servers and the like or to processors in the system), display (e.g., such as display of images or data in graphical and/or text form), and task signal generation (e.g., transmission of instructions to various system components in response to data obtained from other system components to perform certain tasks).

In one aspect, the system processor 18 is used to control voltage differences in the various modules and channels of the system 1. In a preferred aspect, this control is used to increase the amount of time the peptide analysis module 17 actually spends analyzing sample and obtaining sequence information.

Preferably, the system processor 18 can communicate with one or more sensors (e.g., pH sensors, temperature sensors) and/or detectors 23 in communication with the modules and channels of the system 1. Still more preferably, the system processor 18 can modify various system parameters (e.g., reagent flow, voltage) in response to this communication. For example, the output of a detector 23 (e.g., one or more electrical signals) can be processed by the system processor 18 which can perform one or more editing functions. Editing functions include, but are not limited to, removing background, representing signals as images, comparing signals and/or images from duplicate or different runs, performing statistical operations (e.g., such as ensemble averaging as described in Wilm, 1996, supra), and the like. Any of these functions can be performed automatically according to operator-determined criteria, or interactively; i.e., upon displaying an image file to a human operator, the operator can modify various editing menus as appropriate. Preferably, editing menus, for example, in the form of drop-down menus, are displayed on the interface of a user device connectable to the network and in communication with the system 18 processor. Alternatively, or additionally, editing menus can be accessed by selecting one or more icons, radiobuttons, and/or hyperlinks displayed on the interface of the user device.

In a preferred aspect, the processor 18 is capable of implementing a program for inferring the sequence of a protein from a plurality of protein digestion products or unique peptides. Such programs are known in the art and are described in Yates et al., 1991, In Techniques in Protein Chemistry II, by Academic Press, Inc. pp. 477-485; Zhou et al., The 40th ASMS Conference on Mass Spectrometry and Allied Topics, pp. 635-636; and Zhou et al., The 40th ASMS Conference on Mass Spectrometry and Allied Topics, pp. 1396-1397, the entireties of which are incorporated herein by reference.

For example, the system processor 18 can be used to determine all possible combinations of amino acids that can sum to the measured mass of an unknown peptide being analyzed (e.g., by ESI MS/MS) after adjusting for various factors such as water lost in forming peptide bonds, protonation, other factors that alter the measured mass of amino acids, and experimental considerations that constrain the allowed combinations of amino acids. The system processor 18 can then determine linear permutations of amino acids in the permitted combinations. Theoretical fragmentation spectra are then calculated for each permutation and these are compared with an experimental fragmentation spectrum obtained for an unknown peptide to determine the amino acid sequence of the unknown peptide. Many computer programs are commercially available for direct correlation of mass spectral data (product ion spectra) with sequences in protein and nucleotide databases, such as SEQUEST (Thermo Finnigan) and Mascot (Matrix Sciences).

Once an experimentally determined amino acid sequence of an isolated protein or polypeptide fragment thereof has been obtained, the system processor 18 can be used to search available protein databases or nucleic acid sequence databases to determine degree of identity between the protein identified by the system 1 and a sequence in the database Such an analysis may help to characterize the function of the protein. For example, in one aspect, conserved domains within a newly identified protein can be used to identify whether the protein is a signaling protein (e.g., the presence of seven hydrophobic transmembrane regions, an extracellular N-terminus, and a cytoplasmic C-terminus would be a hallmark for a G protein coupled receptor or a GPCR).

Where a database contains one or more partial nucleotide sequences that encode at least a portion of the protein identified by the system 1, such partial nucleotide sequences (or their complement) can serve as probes for cloning a nucleic acid molecule encoding the protein. If no matching nucleotide sequence can be found for the protein identified by the system 1 within a nucleic acid sequence database, a degenerate set of nucleotide sequences encoding the experimentally determined amino acid sequence can be generated which can be used as hybridization probes to facilitate cloning the gene that encodes the protein. Clones thereby obtained can be used to express the protein.

Preferably, the system processor 18 is used to generate a proteome map for a cell. More preferably, the processor 18 also generates proteome maps for the same types of cells in different disease states, for the same types of cells exposed to one or more pathogens or toxins, for the same types of cells during different developmental stages, or is used to compare different types of cells (e.g., from different types of tissues). Maps obtained for cells in a particular disease state can be compared to maps obtained from cells treated with a drug or agent and can be generated for cells at different stages of disease (e.g., for different stages or grades of cancer).

The system processor 18 preferably is used to compare different maps obtained to identify differentially expressed polypeptides in the cells described above. In a preferred aspect, the processor 18 displays the results of such an analysis on the display of a user device, displaying such information as polypeptide name (if known), corresponding amino acid sequence and/or gene sequence, and any expression data (e.g., from genomic analyses) or functional data known. Preferably, data relating to proteome analysis is stored in a database along with any clinical data available relating to patients from whom cells were obtained.

In one aspect, the display comprises a user interface which displays one or more hyperlinks which a user can select to access various portions of the database. In another aspect, processor 18 comprises or is connectable to an information management system which can link the database with other proteomic databases or genomic databases (e.g., such as protein sequence and nucleotide sequence databases).

In a preferred aspect, a proteome map is obtained for a cell comprising a disrupted cell signaling pathway gene and the map is used to identify other polypeptides differentially expressed in the cell (as compared to a cell which comprises a functional cell signaling pathway gene). Differentially expressed proteins are identified as candidate members of the same signaling pathway.

In one aspect, the candidate signaling pathway gene is disrupted in a model system such as a knockout animal (e.g., a mouse) to identify other genes in addition to the candidate signaling pathway gene whose expression is affected by the disruption and which are likely, therefore, to be in the same pathway. Other model systems include, but are not limited to, cell(s) or tissue(s) comprising antisense molecules or ribozymes which prevent translation of an mRNA encoding the candidate polypeptide. Methods of generating such model systems are known in the art. By obtaining proteome maps for multiple disrupted candidate signaling polypeptides, the position of the polypeptides in a pathway can be determined (e.g., to identify whether the polypeptides are upstream or downstream of other pathway polypeptides).

Uses of Cell Signaling Polypeptides

The expression and/or form (e.g., presence or absence of modifications and/or cleavage products or other processed forms) of candidate signaling pathway polypeptides can be evaluated in a plurality of biological samples to evaluate the use of these polypeptides as diagnostic molecules. The expression and/or form (e.g., sequence) of nucleic acid molecules encoding the polypeptides also can be evaluated in the plurality of biological samples as these also may be diagnostic. In a preferred aspect, the biological samples are from patients having a disease (or a particular stage of a disease) or who are at risk of developing a disease. Preferably, the disease is a pathology involving abnormal cell proliferation or cell death (e.g., such as cancer).

When disruption of a candidate signaling pathway polypeptide (e.g., loss of expression, reduced expression, overexpression, ectopic expression of the polypeptide, or the presence of an aberrant form of the polypeptide) is identified as diagnostic of a particular disease or trait, molecular probes reactive with disrupted polypeptide can be contacted with a test sample from a patient suspected of having a disease or trait and reactivity of the molecular probe with the disrupted polypeptide can be determined as a means of determining the presence or absence or risk of having the disease or trait.

In one aspect, the molecular probe is reactive with both the disrupted and non-disrupted polypeptide and the presence of a disrupted polypeptide can be determined by detecting differences in molecular mass or sequence between the disrupted and non-disrupted polypeptide or detecting changes in the quantitative level of a single species of polypeptide (i.e., where the disruption changes the expression rather than the structure of the non-disrupted polypeptide). In another aspect, the molecular probe is specifically reactive with a disrupted form of a polypeptide and does not react with a non-disrupted form of the polypeptide (e.g., the probe reacts with a phosphorylated form of a polypeptide but does not react with a non-phosphorylated form).

Preferably, the probe is an antibody. Polyclonal antisera or monoclonal antibodies can be made using methods known in the art. A mammal such as a mouse, hamster, or rabbit, can be immunized with an immunogenic form of a signaling polypeptide, fragment, modified form thereof, or variant form thereof. Techniques for conferring immunogenicity on such molecules include conjugation to carriers or other techniques well known in the art. For example, the immunogenic molecule can be administered in the presence of adjuvant. Immunization can be monitored by detection of antibody titers in plasma or serum. Standard immunoassay procedures can be used with the immunogen as antigen to assess the levels and the specificity of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (see, e.g., Kohler and Milstein, 1975, Nature 256: 495-497; Kozbor et al., 1983, Immunol. Today 4: 72, Cole et al., 1985, In Monoclonal Antibodies in Cancer Therapy, Allen R. Bliss, Inc., pages 77-96). Additionally, techniques described for the production of single-chain antibodies (U.S. Pat. No. 4,946,778) can be adapted to produce antibodies according to the invention.

Antibody fragments which contain specifically bind to a cell signaling polypeptide, modified forms thereof, and variants thereof, also may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. VH regions and FV regions can be expressed in bacteria using phage expression libraries (e.g., Ward et al., 1989, Nature 341: 544-546; Huse et al., 1989, Science 246: 1275-1281; McCafferty et al., 1990, Nature 348: 552-554).

Chimeric antibodies, i.e., antibody molecules that combine a non-human animal variable region and a human constant region also are within the scope of the invention. Chimeric antibody molecules include, for example, the antigen binding domain from an antibody of a mouse, rat, or other species, with human constant regions. Standard methods may be used to make chimeric antibodies containing the immunoglobulin variable region which recognizes the gene product of cell signaling polypeptides (see, e.g., Morrison et al., 1985, Proc. natl. Acad. Sci. USA 81: 6851; Takeda et al., 1985, Nature 314: 452; U.S. Pat. No. 4,816,567; U.S. Pat. No. 4,816,397). Chimeric antibodies are preferred where the probes are to be used therapeutically to treat a condition associated with physiological responses to an aberrant cell signaling pathways.

Monoclonal or chimeric antibodies can be humanized further by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (see, e.g., Teng et al., 1983, Proc. Natl. Acad. Sci. USA 80: 7308-7312; Kozbor et al., 1983, Immunology Today 4: 7279; Olsson et al., 1982, Meth. Enzymol. 92: 3-16; WO 92/06193; EP 0239400).

In a particularly preferred aspect, an antibody is provided which recognizes a modified and/or variant form of an cell signaling polypeptide but which does not recognize a non-modified and/or non-variant form of the cell signaling polypeptide. For example, peptides comprising the variant region of a variant polypeptide can be used as antigens to screen for antibodies specific for these variants. Similarly modified peptides or proteins can be used as immunogens to select antibodies which bind only to the modified form of the protein and not to the unmodified form. Methods of making variant-specific antibodies and modification-specific antibodies are known in the art and described in U.S. Pat. No. 6,054,273; U.S. Pat. No. 6,054,273; U.S. Pat. No. 6,037,135; U.S. Pat. No. 6,022,683; U.S. Pat. No. 5,702,890; U.S. Pat. No. 5,702,890, and in Sutton et al., 1987, J Immunogenet 14(1): 43-57, for example, the entireties of which are hereby incorporated by reference.

In one aspect, labeled antibodies or antigen-binding portions thereof are provided. Antibodies can be labeled with a fluorescent compound such as fluorescein, amino coumarin acetic acid, tetramethylrhodamine isothiocyanate (TRITC), Texas Red, Cy3.0 and Cy5.0. GFP is also useful for fluorescent labeling, and can be used to label antibodies or antigen-binding portions thereof by expression as fusion proteins. GFP-encoding vectors designed for the creation of fusion proteins are commercially available. Other labels include, but are not limited to, alkaline phosphatase, beta-galactosidase, or acetylcholinesterase; luminescent materials such as luminol; radioactive materials, electron dense substances, such as ferritin or colloidal gold, and other molecules such as biotin.

Polypeptides and/or modified forms thereof and/or variants thereof can be detected using standard immunoassays using the antibodies described above. Immunoassays include, but are not limited to, radioimmunoassays, enzyme immunoassays (e.g. ELISA), immunofluorescence (such as immunohistochemical analyses), immunoprecipitation, latex agglutination, hemagglutination, and histochemical tests. Such assays are routine in the art.

In a particularly preferred aspect of the invention, a plurality of different probes are stably associated at different known locations on a solid support. Preferably, the different probes represent different signaling polypeptides in the same signaling pathway. In one aspect, at least one early pathway probe (i.e., reactive with at least one early pathway polypeptide, downstream of fewer than about 5 pathway polypeptides) and at least one late pathway probe (i.e., reactive with at least one late pathway polypeptide, downstream of greater than about 10 pathway polypeptides). In another aspect, at least about one middle pathway probe is provided (i.e., reactive with at least one middle pathway polypeptide, downstream of greater than about five but less about 10 pathway polypeptides). Preferably, one or more reaction control polypeptides reactive with a constitutively expressed polypeptide (e.g., actin) is provided. One or more background control probes (e.g., reactive with a polypeptide not expected to be in a particular sample, such as a probe reactive with a plant polypeptide where a human sample is evaluated) also is provided. The support and probes can be reacted with a biological sample comprising polypeptides from cell(s) or tissue(s) of a patient (which are preferably labeled) and used to identify cell signaling polypeptides or modified or variant forms thereof expressed in the sample by determining which of the probes on the support react with cellular polypeptides in the sample.

It should be obvious to those of skill in the art that parallel assays can be performed with molecular probes reactive with nucleic acids encoding the cell signaling polypeptides according to the invention. Hybridization-based assays such as Southerns (e.g., to detect deleted or other mutated cell signaling genes), Northerns, RT-PCR, array-based assays and the like (e.g., to detect altered expression of transcripts or the expression of aberrant transcripts corresponding to cell signaling genes identified according to methods of the invention). Such assays are routine in the art.

Cells genetically engineered to express recombinant cell signaling polypeptides according to the invention can be used in a screening program to identify other cellular biomolecules or drugs that specifically interact with the recombinant protein, or to produce large quantities of the recombinant protein, e.g., for therapeutic administration. Possession of cloned genes encoding the cell signaling polypeptides according to the invention permits gene therapy to replace or supplement such polypeptides where the absence or diminished expression of the polypeptides is associated with disease.

### EXAMPLES

The invention will now be further illustrated with reference to the following example. It will be appreciated that what follows is by way of example only and that modifications to detail may be made while still falling within the scope of the invention.

### Example 1

In a particularly preferred embodiment, the system 1 is used to identify a profile of proteins stimulated by PI 3-kinase. Cell lysates are obtained from prostate cancer tissue and from normal prostate tissue from the same or a different patient. Aliquots of lysates are evaluated in parallel using the system 1 to identify differentially expressed proteins while other aliquots are evaluated using nucleic acid arrays (e.g., GeneDevice arrays or cDNA arrays) to identify differentially expressed nucleic acids. Preferably, data obtained from each of these analyses is evaluated using the processor 18 of the system 1.

This analysis can be complemented by an examination of cells in which various proteins in the PI 3 pathway are known to be abnormally activated. For example, the viral form of PI 3-kinase (v-P3k) is constitutively activated, capable of transforming cells in cultures and will induce angiogenesis and hemangiosarcomas in chorioallantoic membrane tissues of embryonated chicks, when introduced via an replication-defective retrovirus. Therefore, in one aspect, v-P3k induced protein expression in cells (e.g., chicken or mouse) transformed in vitro with v-P3k is evaluated to identify proteins that are differentially expressed in these cells as compared to cells that are not transformed. Differentially expressed polypeptides so identified are compared to those differentially expressed in cells obtained from humans. Preferably, the MRNA expression of genes encoding these polypeptides also is determined. As above, protein expression data is preferably evaluated along with nucleic acid expression data. In a particularly preferred aspect, different time points after transformation are evaluated to determine whether particular profiles of protein expression can be correlated with particular physiological responses. For example, where a transformed chicken embryonate is evaluated, protein expression may be correlated with tumor formation or angiogenesis within the chorioallantoic membrane tissues of these embryonates.

v-Src transformed cells (e.g., such as mouse fibroblast cells) are also analyzed using the system 1, since v-Src induces morphological transformation in tissue culture cells and activates a number of downstream signaling proteins, including PI 3-kinase. The well characterized proteins induced by v-Src provide a positive control for the sensitivity of the system 1.

All references, patents, patent applications and patent publications cited herein are hereby incorporated by reference in their entireties. Variations, modifications, and other implementations of what is described herein will occur to those of ordinary skill in the art without departing from the spirit and scope of the present invention as claimed. Accordingly, the present invention is to be defined not by the preceding illustrative description.

We claim:

1. A microfluidic device, comprising:
an inlet channel;
an at least one reaction channel engaged to the inlet channel wherein an enzyme is located within the reaction channel; and
an at least one solid support in communication with the reaction channel capable of concentrating a charged analyte produced by a reaction in the reaction channel.

2. The device of claim 1 wherein the solid support is a membrane.

3. The device of claim 2 wherein the membrane is an ultrafiltration membrane.

4. The device of claim 2 wherein the solid support is a nanocapillary array.

5. The device of claim 2 wherein the at least one membrane comprises a charge.

6. The device of claim 1 wherein the reaction channel comprises a first side channel and a second side channel.

7. The device of claim 6 wherein the first side channel comprises a first charged membrane and the second side channel comprises a second charged membrane.

8. The device of claim 7 where the first charged membrane comprises a positive charge and the second charged membrane comprises a negative charge.

9. The device of claim 8 further comprising a negative electrode adjacent to the positive charged membrane in the first side channel.

10. The device of claim 9 further comprising a positive electrode adjacent to the negatively charged membrane in the second side channel.

11. The device of claim 1 further comprising an upstream separation module which delivers a substantially purified polypeptide to the microfluidic device.

12. The device of claim 1 further comprising a downstream separation module in communication with an outlet channel of the microfluidic device.

13. A microfluidic device, comprising:
a first cover channel slide comprising an input channel and an output channel;
a second cover channel side located beneath the first cover channel slide comprising a first channel aligned beneath the input channel and a second channel aligned beneath the output channel;
a nanocapillary array located between the first cover channel slide and the second channel cover slide; and
a bottom slide located beneath the second channel cover slide.

14. The device of claim 13 wherein the inlet channel is in communication with an upstream separation module wherein the upstream separation module delivers a substantially purified polypeptide to the microfluidic device.

15. The device of claim 13 wherein the outlet channel is in communication with a downstream separation module.

16. The device of claim 13 wherein a sample is concentrated at the nanocapillary array.

17. The device of claim 13 wherein the nanocapillary array is an ultrafiltration membrane.

18. The device of claim 13 wherein the microfluidic device comprises a protease.

19. A method of concentrating analytes, comprising:
delivering a substantially purified polypeptide to a microfluidic device;
reacting a the substantially purified polypeptide with an enzyme within a reaction channel of the microfluidic device wherein a positive analyte and a negative analyte are produced;
concentrating the positive analyte at a first membrane;
concentrating the negative analyte at a second membrane; and
removing the concentrated analytes from the microfluidic device.

20. The method of claim 19 wherein the positive analyte is concentrated at a positively charged membrane.

21. The method of claim 19 wherein the negative analyte is concentrated at a negatively charged membrane.

22. The method of claim 19 further comprising engaging an upstream separation module to the microfluidic device wherein the upstream separation module produces the substantially purified polypeptide.

23. The method of claim 19 further comprising delivering the concentrated analyte to a down stream separation module after being removed from the microfluidic device.

24. The method of claim 19 further comprising utilizing electroosmotic flow to move the charged analytes.

25. The method of claim 19 further comprising providing a first side channel compring a positively charged membrane.

26. The method of claim 25 further comprising positioning a negatively charged electrode adjacent to the positively charged membrane.

27. The method of claim 26 further comprising positioning a positively charged electrode adjacent to the negatively charged membrane.

28. A method of analyzing a substantially purified polypeptide, comprising:
delivering a substantially purified polypeptide to a microfluidic device;
delivering a first portion of the substantially purified polypeptide to a reaction chamber of the microfluidic device and delivering a second portion of the substantially purified polypeptide to a peptide analysis module;
reacting the first portion of the substantially purified polypeptide with an agent wherein a reaction product is produced;
delivering the reaction product to the peptide analysis module;
perform a test on the second portion of the substantially purified polypeptide to obtain a first test result;
perform the test on the reaction product to obtain a second test result; and
compare the first test result with the second test result.

29. The method of claim 28 wherein the agent is an enzyme.

30. The method of claim 29 wherein the enzyme is a phosphatase.

31. The method of claim 29 wherein the enzyme is a protease.

32. The method of claim 28 wherein the agent is a derivatization agent.

33. The method of claim 29 wherein the enzyme is a cross-linking enzyme.

34. The method of claim 28 further comprising delivering an at least third portion of the substantially purified polypeptide to an at least second reaction channel.

35. The method of claim 28 further comprising providing an upstream separation module capable of producing the substantially purified polypeptide wherein the upstream separation module delivers the substantially purified polypeptide to the microfluidic device.
